SEQUENCE LISTING (79 sequences)

SEQ ID NO: 1 - DNA, 88 bp, Artificial Sequence
  oligonucleotide primer
aaaccacgtcctcgtcacacgtcgttcgcggacatagcaagaaatccacgtcgccacatctcgagacgatttattcaacaaagccacg

SEQ ID NO: 2 - DNA, 88 bp, Artificial Sequence
  oligonucleotide primer
aacggcgtcaggtctttgggactcatgacgcgcggttttcaaaattccctgcgcgcgcgacgggcgccagtgttacaaccaattaacc

SEQ ID NO: 3 - DNA, 81 bp, Artificial Sequence
  oligonucleotide primer
gcgccacacgcccggagcctcgagttcagcgtgcggctctttgccaactagcctgcgtcacggcgatttattcaacaaagc

SEQ ID NO: 4 - DNA, 88 bp, Artificial Sequence; identical to SEQ ID NO: 2
  oligonucleotide primer

SEQ ID NO: 5 - DNA, 82 bp, Artificial Sequence
  oligonucleotide primer
tgcgttctgtggtgcgtctggatctgtctctcgacgtttctgatagccatgttccatcgacgatttattcaacaaagccacg

SEQ ID NO: 6 - DNA, 81 bp, Artificial Sequence
  oligonucleotide primer
cggcacacatccagccgtttgtgtttcttaacgctctccaggtactgatccaggcccacggccagtgttacaaccaattaa

SEQ ID NO: 7 - DNA, 81 bp, Artificial Sequence
  oligonucleotide primer
ttgagatttctgtcgccgactaaattcatgtcgcgcgatagtggtgtttatcgccgatagcgatttattcaacaaagccac

SEQ ID NO: 8 - DNA, 81 bp, Artificial Sequence
  oligonucleotide primer
aatattgatttacgctatataaccaatgactaatatggctaatggccaatattgatgcaagccagtgttacaaccaattaa

SEQ ID NO: 9 - DNA, 81 bp, Artificial Sequence
  oligonucleotide primer
gactatgtgcatgttcggctactgagctaccgaggcgaccccctggtcttcaagcacactcgatttattcaacaaagccac

SEQ ID NO: 10 - DNA, 81 bp, Artificial Sequence
  oligonucleotide primer
caccaggtaggttatcaaaacgcgagcccatatcgccgccatcattgtaatcagcaatgtgccagtgttacaaccaattaa

SEQ ID NO: 11 - DNA, 81 bp, Artificial Sequence
  oligonucleotide primer
acgtcctcgtcacacgtcgttcgcggacatagcaagaaattcacgtcgccacgtctcgagacgatttattcaacaaagcca

SEQ ID NO: 12 - DNA, 84 bp, Artificial Sequence
  oligonucleotide primer
aaggttcttccatttccgaggcggtcagttcatcgtacaccgagacgtagtacctgatggggccagtgttacaaccaattaacc

SEQ ID NO: 13 - DNA, 76 bp, Artificial Sequence
  oligonucleotide primer
tgtctttcggttccaactctttccccgccccatcacctcgcctgtactatgtgtcgatttattcaacaaagccacg

SEQ ID NO: 14 - DNA, 87 bp, Artificial Sequence
  oligonucleotide primer
tcgcgcgacatgaatttagtcggcgacagaaatctcgaaacgcgtatttcggacaaacacacatgccagtgttacaaccaattaacc

SEQ ID NO: 15 - DNA, 90 bp, Artificial Sequence
  oligonucleotide primer
cgctgtagggataaatagtgcgatggcgtttgtgggagaacgcagtagcgatgggttgcgacgtgcaccgatttattcaacaaagccacg

SEQ ID NO: 16 - DNA, 81 bp, Artificial Sequence; identical to SEQ ID NO: 10
  oligonucleotide primer

SEQ ID NO: 17 - DNA, 81 bp, Artificial Sequence
  oligonucleotide primer
cagtctgcaacatgcggctgtgctgggtgtggctgtctgtttgtctgtgcgccgtggtgccgatttattcaacaaagccac

SEQ ID NO: 18 - DNA, 74 bp, Artificial Sequence
  oligonucleotide primer
gctagttggcaaagagccgcacgctgaactcgaggctccgggcgtgtggcggccagtgttacaaccaattaacc

SEQ ID NO: 19 - DNA, 83 bp, Artificial Sequence
  oligonucleotide primer
gattttccgggaataccggatattacgaattactggtagtgacgtagataataaaattatacgatttattcaacaaagccacg

SEQ ID NO: 20 - DNA, 76 bp, Artificial Sequence
  oligonucleotide primer
caccaaagccgttagcgtgcccagagctaccgcacggtaaaatagggacatgagccagtgttacaaccaattaacc

SEQ ID NO: 21 - DNA, 408 bp, Human cytomegalovirus
gtctgcaacatgcggctgtctcgggtgtggctgtctgtttgtctgtgcgccgtggtgctgggtcagtgccagcgggagaccgcagaaaaaaacgattattaccgagtaccgcattactgggacgcgtgctctcgcgcgctgcctgaccaaacccgttacaagtatgtggaacagctcgtggacctcacgttgaactaccactacgatgcgagccacggcttggacaactttgacgtgctcaagagaatcaacgtgaccgaggtgtcgttgctcatcagcgactttatacgtcagaaccgtcgcggcggcaccaacaaaaggaccacgttcaacgccgccggttcgctggcgcctcacgcccggagcctcgagttcagcgtgcggctctttgccaactagcctgcgtca

SEQ ID NO: 22 - DNA, 516 bp, Human cytomegalovirus
gtctgcaacatgcggctgtgtcgggtgtggctgtctgtttgtctgtgcgccgtggtgctgggtcagtgccagcgggagaccgcagaaaaaaacgattattaccgagtaccgcattactgggacgcgtgctctcgcgcgctgcctgaccaaacccgttacaagtatgtggaacagctcgtggacctcacgttgaactaccactacgatgcgagccacggcttggacaactttgacgtgctcaagaggtgagggtacgcgctaaaggtgtatgacaacgggaaggtaagggcgaacgggtaacgggtaggtaaccgcatggggtgtgaaatgacgttcggaacctgtgcttgcagaatcaacgtgaccgaggtgtcgttgctcatcagcgactttagacgtcagaaccgtcgcggcggcaccaacaaaaggaccacgttcaacgccgccggttcgctggcgcctcacgcccggagcctcgagttcagcgtgcggctctttgccaactagcctgcgtca

SEQ ID NO: 23 - DNA, 410 bp, Human cytomegalovirus
gtctgcaacatgcggctgtgtcgggtgtggctgtctgtttgtctgtgcgccgtggtgctgggtcagtgccagcgggagaccgcagaaaaaaaaacgattattaccgagtaccgcattactgggacgcgtgctctcgcgcgctgcctgaccaaacccgttacaagtatgtggaacagctcgtggacctcacgttgaactaccactacgatgcgagccacggcttggacaactttgacgtgctcaagagaatcaacgtgaccgaggtgtcgttgctcatcagcgactttagacgtcagaaccgtcgcggcggcaccaacaaaaggaccacgttcaacgccgccggttcgctggcgcctcacgcccggagcctcgagttcagcgtgcggctctttgccaactagcctgcgtca

SEQ ID NO: 24 - DNA, 516 bp, Human cytomegalovirus; identical to SEQ ID NO: 22

SEQ ID NO: 25 - DNA, 656 bp, Human cytomegalovirus
ccgtgcgtcatgagtcccaaaaacctgacgccgttcttgacggcgttgtggctgctattgggtcacagccgcgtgccgcgggtacgcgcagaagaatgttgcgaattcataaacgtcaaccacccgccggaacgctgttacgatttcaaaatgtgcaatcgcttcaccgtcgcgtacgtattttcatgattgtctgcgttctgtggtgcgtctggatctgtctctcgacgtttctgatagccatgttccatcgacgatcctcgggaatgccagagtagattttcatgaatccacaggctgcggtgtccggacggcgaagtctgctacagtcccgagaaaacggctgagattcgcgggatcgtcaccaccatgacccattcattgacacgccaggtcgtacacaacaaactgacgaactgcaactacaatccgttatacctcgaagctgacgggcgaatacgctgcggcaaagtgaacgacaaggcgcagtacctgctgggcgccgctggcagcgttccctatcgatggatcaacctggaatacgacaagataacccggatcgtgggcctggatcagtacctggagagcgttaagaaacacaaacggctggatgtgtgccgcgctaaaatgggctatatgctgcagtgaataataaa

SEQ ID NO: 26 - DNA, 656 bp, Human cytomegalovirus
ccgcgcgtcatgagtcccaaaaacctgacgccgttcttgacggcgttgtggctgctattgggtcacagccgcgtgccgcgggtacgcgcagaagaatgttgcgaattcataaacgtcaaccacccgccggaacgctgttacgatttcaaaatgtgcaatcgcttcaccgtcgcgtacgtattttcatgattgtctgcgttctgtggtgcgtctggatctgtctctcgacgtttctgatagccatgttccatcgacgatcctcgggaatgccagagtagattttcatgaatccacaggctgcggtgtccggacggcgaagtctgctacagtcccgagaaaacggctgagattcgcgggatcgtcaccaccatgacccattcattgacacgccaggtcgtacacaacaaactgacgagctgcaactacaatccgttatacctcgaagctgacgggcgaatacgctgcggcaaagtgaacgacaaggcgcagtacctgctgggcgccgctggcggcgttccctatcgatggatcaacctggaatacgacaagatagcccggatcgtgggcctggatcagtacctggagagcgttaagaaacacaaacggctggatgtgtgccgcgctaaaatgggctatatgctgcagtgaataataaa

SEQ ID NO: 27 - DNA, 533 bp, Human cytomegalovirus
ccgcgcgtcatgagtcccaaaaacctgacgccgttcttgacggcgttgtggctgctattgggtcacagccgcgtgccgcgggtacgcgcagaagaatgttgcgaattcataaacgtcaaccacccgccggaacgctgttacgatttcaaaatgtgcaatcgcttcaccgtcgcgctgcggtgtccggacggcgaagtctgctacagtcccgagaaaacggctgagattcgcgggatcgtcaccaccatgacccattcattgacacgccaggtcgtacacaacaaactgacgagctgcaactacaatctgttatacctcgaagctgacgggcgaatacgctgcggcaaagtgaacgacaaggcgcagtacctgctgggcgccgctggcagcgttccctatcgatggatcaacctggaatacgacaagataacccggatcgtgggcctggatcagtacctggagagcgttaagaaatacaaacggctggatgtgtgccgcgctaaaatgggctatatgctgcagtgaataataaa

SEQ ID NO: 28 - DNA, 775 bp, Human cytomegalovirus
ccgcgcgtcatgagtcccaaaaacctgacgccgttcttgacggcgttgtggctgctattgggtcacagccgcgtgccgcgggtacgcgcagaagaatgttgcgaattcataaacgtcaaccacccgccggaacgctgttacgatttcaaaatgtgcaatcgcttcaccgtcgcgtacgtatttttatgattgtctgcgttctgtggtgcgtctggatttgtctctcgacgtttctgatagccatgttccatcgacgatcctcgggaatgccagagtagattttcatgaatccacaggctgcggtgtccggacggcgaagtctgctacagtcccgagaaaacggctgagattcgcgggatcgtcaccaccatgacccattcattgacacgccaggtcgtacacaacaaactgacgagctgcaactacaatccgtaagtctcttcctcgagggccttacagcctatgggaaagtaagacagagggacaaaacatcattaaaaaaaaagtctaatttcacgttttgtacccccccttcccctccgtgttgtaggttatacctcgaagctgacgggcgaatacgctgcggcaaagtgaacgacaaggcgcagtacctgctgggcgccgctggcagcgttccctatcgatggatcaacctggaatacgacaagataacccggatcgtgggcctggatcagtacctggagagcgttaagaaacacaaacggctggatgtgtgccgcgctaaaatgggctatatgctgcagtgaataataaa

SEQ ID NO: 29 - DNA, 60 bp, Human cytomegalovirus
cgctaaaatgggctatatgctgcagtgaataataaaatgtgtgtttgtccgcaaaaaaaa

SEQ ID NO: 30 - DNA, 60 bp, Human cytomegalovirus
cgctaaaatgggctatatgctgcagtgaataataaaatgtgtgtttgtccaaaaaaaaaa

SEQ ID NO: 31 - DNA, 60 bp, Human cytomegalovirus; identical to SEQ ID NO: 30

SEQ ID NO: 32 - DNA, 52 bp, Human cytomegalovirus
cgctaaaatgggctatatgctgcagtgaataataaaatgtgtgtttgtccga

SEQ ID NO: 33 - DNA, 1977 bp, Human cytomegalovirus
gtctgcaacatgcggctgtgtcgggtgtggctgtctgtttgtctgtgcgccgtggtgctgggtcagtgccagcgggagaccgcagaaaaaaacgattattaccgagtaccgcattactgggacgcgtgctctcgcgcgctgcctgaccaaacccgttacaagtatgtggaacagctcgtggacctcacgttgaactaccactacgatgcgagccacggcttggacaactttgacgtgctcaagaggtgagggtacgcgctaaaggtgtatgacaacgggaaggtaagggcgaacgggtaacgggtaggtaaccgcatggggtgtgaaatgacgttcggaacctgtgcttgcagaatcaacgtgaccgaggtgtcgttgctcatcagcgactttagacgtcagaaccgtcgcggcggcaccaacaaaaggaccacgttcaacgccgccggttcgctggcgcctcacgcccggagcctcgagttcagcgtgcggctctttgccaactagcctgcgtcacgggaaataatatgctacggcttctgcttcgtcaccactttcactgcctgcttctgtgcgcggtttgggcaacgccctgtctggcgtctccgtggttcacgctaacggcgaaccagaatccgtccccgccatggtctaaactgacgtatcccaaaccgcatgacgcggcgacgttttactgtccttttctctatccctcgcccccacggtccccctcgcaattcccggggttccagcgggtatcaacgggtcccgagtgtcgcaacgagaccctgtatctgctgtacaaccgggaaggccagaccttggtggagagaagctccacctgggtgaaaaaggtgatctggtatctgagcggtcgcaatcagaccatcctccaacggatgccccgaacggcttcgaaaccgagcgacggaaacgtgcagatcagcgtggaagacgccaagatttttggagcgcacatggtgcccaagcagaccaagctgctacgtttcgtcgtcaacgatggcacacgttatcagatgtgtgtgatgaaactggagagctgggcccacgtcttccgggactacagcgtgtcttttcaggtgcgattgacgttcaccgaggccaataaccagacttacaccttctgcacccatcccaatctcatcgtttgagcccgtcgcgcgcgcagggaattttgaaaaccgcgcgtcatgagtcccaaaaacctgacgccgttcttgacggcgttgtggctgctattgggtcacagccgcgtgccgcgggtacgcgcagaagaatgttgcgaattcataaacgtcaaccacccgccggaacgctgttacgatttcaaaatgtgcaatcgcttcaccgtcgcgtacgtattttcatgattgtctgcgttctgtggtgcgtctggatttgtctctcgacgtttctgatagccatgttccatcgacgatcctcgggaatgccagagtagattttcatgaatccacaggctgcggtgtccggacggcgaagtctgctacagtcccgagaaaacggctgagattcgcgggatcgtcaccaccatgacccattcattgacacgccaggtcgtacacaacaaactgacgagctgcaactacaatccgtaagtctcttcctcgagggccttacagcctatgggaaagtaagacagagggacaaaacatcattaaaaaaaaagtctaatttcacgttttgtacccccccttcccctccgtgttgtaggttatacctcgaagctgacgggcgaatacgctgcggcaaagtgaacgacaaggcgcagtacctgctgggcgccgctggcagcgttccctatcgatggatcaacctggaatacgacaagataacccggatcgtgggcctggatcagtacctggagagcgttaagaaacacaaacggctggatgtgtgccgcgctaaaatgggctatatgctgcagtgaataataaa

SEQ ID NO: 34 - protein, 129 aa, Human cytomegalovirus
MRLCRVWLSVCLCAVVLGQCQRETAEKNDYYRVPHYWDACSRALPDQTRYKYVEQLVDLTLNYHYDASHGLDNFDVLKRINVTEVSLLISDFRRQNRRGGTNKRTTFNAAGSLAPHARSLEFSVRLFAN

SEQ ID NO: 35 - DNA, 30 bp, Artificial Sequence
  oligonucleotide primer
cggcacacatccagccgtttgtgtttctta

SEQ ID NO: 36 - DNA, 30 bp, Artificial Sequence
  oligonucleotide primer
taacgctctccaggtactgatccaggccca

SEQ ID NO: 37 - DNA, 30 bp, Artificial Sequence
  oligonucleotide primer
tcgtcagtttgttgtgtacgacctggcgtg

SEQ ID NO: 38 - DNA, 30 bp, Artificial Sequence
  oligonucloetide primer
tattggcctcggtgaacgtcaatcgcacct

SEQ ID NO: 39 - DNA, 30 bp, Artificial Sequence
  oligonucleotide primer
tgtgtcgggtgtggctgtctgtttgtctgt

SEQ ID NO: 40 - DNA, 30 bp, Artificial Sequence
  oligonucleotide primer
tctgcttcgtcaccactttcactgcctgct

SEQ ID NO: 41 - DNA, 30 bp, Artificial Sequence
  oligonucleotide primer
cgcagaagaatgttgcgaattcataaacgt

SEQ ID NO: 42 - DNA, 30 bp, Artificial Sequence
  oligonulceotide primer
gctgcggtgtccggacggcgaagtctgcta

SEQ ID NO: 43 - DNA, 30 bp, Artificial Sequence
  oligonucleotide primer
ccagctggcagattcccaaactaatgaaag

SEQ ID NO: 44 - DNA, 30 bp, Artificial Sequence
  oligonucleotide primer
ctttcggttccaactctttccccgccccat

SEQ ID NO: 45 - DNA, 30 bp, Artificial Sequence
  oligonucleotide primer
cacctcgcctatactatgtgtatgatgtct

SEQ ID NO: 46 - DNA, 30 bp, Artificial Sequence
  oligonucleotide primer
ctctctttctcagtctgcaacatgcggctg

SEQ ID NO: 47 - DNA, 30 bp, Artificial Sequence
  oligonucleotide primer
gttgtccaagccgtcgctcgcatcgtagtg

SEQ ID NO: 48 - DNA, 30 bp, Artificial Sequence
  oligonucleotide primer
cataataaagctctctttctcagtctgcaa

SEQ ID NO: 49 - DNA, 30 bp, Artificial Sequence
  oligonucleotide primer
tatgatgtctcataataaagctctctttct

SEQ ID NO: 50 - DNA, 390 bp, Human cytomegalovirus
atgcggctgtctcgggtgtggctgtctgtttgtctgtgcgccgtggtgctgggtcagtgccagcgggagaccgcagaaaaaaacgattattaccgagtaccgcattactgggacgcgtgctctcgcgcgctgcctgaccaaacccgttacaagtatgtggaacagctcgtggacctcacgttgaactaccactacgatgcgagccacggcttggacaactttgacgtgctcaagagaatcaacgtgaccgaggtgtcgttgctcatcagcgactttatacgtcagaaccgtcgcggcggcaccaacaaaaggaccacgttcaacgccgccggttcgctggcgcctcacgcccggagcctcgagttcagcgtgcggctctttgccaactag

SEQ ID NO: 51 - protein, 129 aa, Human cytomegalovirus
MRLSRVWLSVCLCAVVLGQCQRETAEKNDYYRVPHYWDACSRALPDQTRYKYVEQLVDLTLNYHYDASHGLDNFDVLKRINVTEVSLLISDFIRQNRRGGTNKRTTFNAAGSLAPHARSLEFSVRLFAN

SEQ ID NO: 52 - DNA, 240 bp, Human cytomegalovirus
atgcggctgtgtcgggtgtggctgtctgtttgtctgtgcgccgtggtgctgggtcagtgccagcgggagaccgcagaaaaaaacgattattaccgagtaccgcattactgggacgcgtgctctcgcgcgctgcctgaccaaacccgttacaagtatgtggaacagctcgtggacctcacgttgaactaccactacgatgcgagccacggcttggacaactttgacgtgctcaagaggtga

SEQ ID NO: 53 - protein, 79 aa, Human cytomegalovirus
MRLCRVWLSVCLCAVVLGQCQRETAEKNDYYRVPHYWDACSRALPDQTRYKYVEQLVDLTLNYHYDASHGLDNFDVLKR

SEQ ID NO: 54 - DNA, 1977 bp, Human cytomegalovirus; identical to SEQ ID NO: 33

SEQ ID NO: 55 - DNA, 1741 bp, Human cytomegalovirus
atgcggctgtctcgggtgtggctgtctgtttgtctgtgcgccgtggtgctgggtcagtgccagcgggagaccgcagaaaaaaacgattattaccgagtaccgcattactgggacgcgtgctctcgcgcgctgcctgaccaaacccgttacaagtatgtggaacagctcgtggacctcacgttgaactaccactacgatgcgagccacggcttggacaactttgacgtgctcaagagaatcaacgtgaccgaggtgtcgttgctcatcagcgactttatacgtcagaaccgtcgcggcggcaccaacaaaaggaccacgttcaacgccgccggttcgctggcgcctcacgcccggagcctcgagttcagcgtgcggctctttgccaactagcctgcgtcacgggaaataatatgctacggcttctgcttcgtcaccactttcactgcctgcttctgtgcgcggtttgggcaacgccctgtctggcgtctccgtggttcacgctaacggcgaaccagaatccgtccccgccatggtctaaactgacgtatcccaaaccgcatgacgcggcgacgttttactgtccttttctctatccctcgcccccacggtccccctcgcaattcccggggttccagcgggtatcaacgggtcccgagtgtcgcaacgagaccctgtatctgctgtacaaccgggaaggccagaccttggtggagagaagctccacctgggtgaaaaaggtgatctggtatctgagcggtcgcaatcagaccatcctccaacggatgccccgaacggcttcgaaaccgagcgacggaaacgtgcagatcagcgtggaagacgccaagatttttggagcgcacatggtgcccaagcagaccaagctgctacgtttcgtcgccaacgatggcacacgttatcagatgtgtgtgatgaaactggagagctgggcccacgtcttccgggactacagcgtgtcttttcaggtgcgattgacgttcaccgaggccaataaccagacttacaccttctgcacccatcccaatctcatcgtttgagcccgtcgcgcgcgcagggaattttgaaaaccgtgcgtcatgagtcccaaaaacctgacgccgttcttgacggcgttgtggctgctattgggtcacagccgcgtgccgcgggtacgcgcagaagaatgttgcgaattcataaacgtcaaccacccgccggaacgctgttacgatttcaaaatgtgcaatcgcttcaccgtcgcgtacgtattttcatgattgtctgcgttctgtggtgcgtctggatctgtctctcgacgtttctgatagccatgttccatcgacgatcctcgggaatgccagagtagattttcatgaatccacaggctgcggtgtccggacggcgaagtctgctacagtcccgagaaaacggctgagattcgcgggatcgtcaccaccatgacccattcattgacacgccaggtcgtacacaacaaactgacgaactgcaactacaatccgttatacctcgaagctgacgggcgaatacgctgcggcaaagtgaacgacaaggcgcagtacctgctgggcgccgctggcagcgttccctatcgatggatcaacctggaatacgacaagataacccggatcgtgggcctggatcagtacctggagagcgttaagaaacacaaacggctggatgtgtgccgcgctaaaatgggctatatgctgcagtgaataataaa

SEQ ID NO: 56 - DNA, 390 bp, Human cytomegalovirus; identical to SEQ ID NO: 50

SEQ ID NO: 57 - protein, 129 aa, Human cytomegalovirus; identical to SEQ ID NO: 51

SEQ ID NO: 58 - DNA, 1977 bp, Human cytomegalovirus; identical to SEQ ID NO: 33

SEQ ID NO: 59 - DNA, 1849 bp, Human cytomegalovirus
atgcggctgtgtcgggtgtggctgtctgtttgtctgtgcgccgtggtgctgggtcagtgccagcgggagaccgcagaaaaaaacgattattaccgagtaccgcattactgggacgcgtgctctcgcgcgctgcctgaccaaacccgttacaagtatgtggaacagctcgtggacctcacgttgaactaccactacgatgcgagccacggcttggacaactttgacgtgctcaagaggtgagggtacgcgctaaaggtgtatgacaacgggaaggtaagggcgaacgggtaacgggtaggtaaccgcatggggtgtgaaatgacgttcggaacctgtgcttgcagaatcaacgtgaccgaggtgtcgttgctcatcagcgactttagacgtcagaaccgtcgcggcggcaccaacaaaaggaccacgttcaacgccgccggttcgctggcgcctcacgcccggagcctcgagttcagcgtgcggctctttgccaactagcctgcgtcacgggaaataatatgctacggcttctgcttcgtcaccactttcactgcctgcttctgtgcgcggtttgggcaacgccctgtctggcgtctccgtggttcacgctaacggcgaaccagaatccgtccccgccatggtctaaactgacgtatcccaaaccgcatgacgcggcgacgttttactgtccttttctctatccctcgcccccacggtccccctcgcaattcccggggttccagcgggtatcaacgggtcccgagtgtcgcaacgagaccctgtatctgctgtacaaccgggaaggccagaccttggtggagagaagctccacctgggtgaaaaaggtgatctggtatctgagcggtcgcaatcagaccatcctccaacggatgccccgaacggcttcgaaaccgagcgacggaaacgtgcagatcagcgtggaagacgccaagatttttggagcgcacatggtgcccaagcagaccaagctgctacgtttcgtcgtcaacgatggcacacgttatcagatgtgtgtgatgaaactggagagctgggcccacgtcttccgggactacagcgtgtcttttcaggtgcgattgacgttcaccgaggccgataaccagacttacaccttctgcacccatcccaatctcatcgtttgagcccgtcgcgcgcgcagggaattttgaaaaccgcgcgtcatgagtcccaaaaacctgacgccgttcttgacggcgttgtggctgctattgggtcacagccgcgtgccgcgggtacgcgcagaagaatgttgcgaattcataaacgtcaaccacccgccggaacgctgttacgatttcaaaatgtgcaatcgcttcaccgtcgcgtacgtattttcatgattgtctgcgttctgtggtgcgtctggatctgtctctcgacgtttctgatagccatgttccatcgacgatcctcgggaatgccagagtagattttcatgaatccacaggctgcggtgtccggacggcgaagtctgctacagtcccgagaaaacggctgagattcgcgggatcgtcaccaccatgacccattcattgacacgccaggtcgtacacaacaaactgacgagctgcaactacaatccgttatacctcgaagctgacgggcgaatacgctgcggcaaagtgaacgacaaggcgcagtacctgctgggcgccgctggcggcgttccctatcgatggatcaacctggaatacgacaagatagcccggatcgtgggcctggatcagtacctggagagcgttaagaaacacaaacggctggatgtgtgccgcgctaaaatgggctatatgctgcagtgaataataaa

SEQ ID NO: 60 - DNA, 240 bp, Human cytomegalovirus; identical to SEQ ID NO: 52

SEQ ID NO: 61 - protein, 79 aa, Human cytomegalovirus; identical to SEQ ID NO: 53

SEQ ID NO: 62 - DNA, 180 bp, Human cytomegalovirus
atgagtcccaaaaacctgacgccgttcttgacggcgttgtggctgctattgggtcacagccgcgtgccgcgggtacgcgcagaagaatgttgcgaattcataaacgtcaaccacccgccggaacgctgttacgatttcaaaatgtgcaatcgcttcaccgtcgcgtacgtattttcatga

SEQ ID NO: 63 - protein, 59 aa, Human cytomegalovirus
MSPKNLTPFLTALWLLLGHSRVPRVRAEECCEFINVNHPPERCYDFKMCNRFTVAYVFS

SEQ ID NO: 64 - DNA, 515 bp, Human cytomegalovirus
atgagtcccaaaaacctgacgccgttcttgacggcgttgtggctgctattgggtcacagccgcgtgccgcgggtacgcgcagaagaatgttgcgaattcataaacgtcaaccacccgccggaacgctgttacgatttcaaaatgtgcaatcgcttcaccgtcgcactgcggtgtccggacggcgaagtctgctacagtcccgagaaacggctgagattcgcgggatcgtcaccaccatgacccattcattgacacgccaggtcgtacacaacaaactgacgagctgcaactacaatctgttatacctcgaagctgacgggcgaatacgctgcggcaaagtgaacgacaaggcgcagtacctgctgggcgccgctggcagcgttccctatcgatggatcaacctggaatacgacaagataacccggatcgtgggcctggatcagtacctggagagcgttaagaaacacaaacggctggatgtgtgccgcgctaaaatgggctatatgctgcagtga

SEQ ID NO: 65 - protein, 171 aa, Human cytomegalovirus
MSPKNLTPFLTALWLLLGHSRVPRVRAEECCEFINVNHPPERCYDFKMCNRFTVALRCPDGEVCYSPEKTAEIRGIVTTMTHSLTRQVVHNKLTSCNYNLLYLEADGRIRCGKVNDKAQYLLGAAGSVPYRWINLEYDKITRIVGLDQYLESVKKHKRLDVCRAKMGYMLQ

SEQ ID NO: 66 - DNA, 804 bp, Human cytomegalovirus
atgagtcccaaaaacctgacgccgttcttgacggcgttgtggctgctattgggtcacagccgcgtgccgcgggtacgcgcagaagaatgttgcgaattcataaacgtcaaccacccgccggaacgctgttacgatttcaaaatgtgcaatcgcttcaccgtcgcgtacgtattttcatgattgtctgcgttctgtggtgcgtctggatctgtctctcgacgtttctgatagccatgttccatcgacgatcctcgggaatgccagagtagattttcatgaatccacaggctgcggtgtccggacggcgaagtctgctacagtcccgagaaaacggctgagattcgcgggatcgtcaccaccatgacccattcattgacacgccaggtcgtacacaacaaactgacgagctgcaactacaatccgtaagtctcttcctcgagggccttacagcctatgggaaagtaagacagagggacaaaacatcattaaaaaaaaagtctaatttcacgttttgtacccccccttcccctccgtgttgtaggttatacctcgaagctgacgggcgaatacgctgcggcaaagtgaacgacaaggcgcagtacctgctgggcgccgctggcggcgttccctatcgatggatcaacctggaatacgacaagatagcccggatcgtgggcctggatcagtacctggagagcgttaagaaacacaaacggctggatgtgtgccgcgctaaaatgggctatatgctgcagtgaataataaaatgtgtgtttgtccgaaatacgcgttttgagatttctg

SEQ ID NO: 67 - DNA, 685 bp, Human cytomegalovirus
atgagtcccaaaaacctgacgccgttcttgacggcgttgtggctgctattgggtcacagccgcgtgccgcgggtacgcgcagaagaatgttgcgaattcataaacgtcaaccacccgccggaacgctgttacgatttcaaaatgtgcaatcgcttcaccgtcgcgtacgtattttcatgattgtctgcgttctgtggtgcgtctggatctgtctctcgacgtttctgatagccatgttccatcgacgatcctcgggaatgccagagtagattttcatgaatccacaggctgcggtgtccggacggcgaagtctgctacagtcccgagaaaacggctgagattcgcgggatcgtcaccaccatgacccattcattgacacgccaggtcgtacacaacaaactgacgagctgcaactacaatccgttatacctcgaagctgacgggcgaatacgctgcggcaaagtgaacgacaaggcgcagtacctgctgggcgccgctggcggcgttccctatcgatggatcaacctggaatacgacaagatagcccggatcgtgggcctggatcagtacctggagagcgttaagaaacacaaacggctggatgtgtgccgcgctaaaatgggctatatgctgcagtgaataataaaatgtgtgtttgtccaaaaaaaaaaaaaaaaaaaaaaaa

SEQ ID NO: 68 - DNA, 180 bp, Human cytomegalovirus; identical to SEQ ID NO: 62

SEQ ID NO: 69 - protein, 59 aa, Human cytomegalovirus; identical to SEQ ID NO: 63

SEQ ID NO: 70 - DNA, 780 bp, Human cytomegalovirus
atgagtcccaaaaacctgacgccgttcttgacggcgttgtggctgctattgggtcacagccgcgtgccgcgggtacgcgcagaagaatgttgcgaattcataaacgtcaaccacccgccggaacgctgttacgatttcaaaatgtgcaatcgcttcaccgtcgcgtacgtatttttatgattgtctgcgttctgtggtgcgtctggatttgtctctcgacgtttctgatagccatgttccatcgacgatcctcgggaatgccagagtagattttcatgaatccacaggctgcggtgtccggacggcgaagtctgctacagtcccgagaaaacggctgagattcgcgggatcgtcaccaccatgacccattcattgacacgccaggtcgtacacaacaaactgacgagctgcaactacaatccgtaagtctcttcctcgagggccttacagcctatgggaaagtaagacagagggacaaaacatcattaaaaaaaaagtctaatttcacgttttgtacccccccttcccctccgtgttgtaggttatacctcgaagctgacgggcgaatacgctgcggcaaagtgaacgacaaggcgcagtacctgctgggcgccgctggcagcgttccctatcgatggatcaacctggaatacgacaagataacccggatcgtgggcctggatcagtacctggagagcgttaagaaacacaaacggctggatgtgtgccgcgctaaaatgggctatatgctgcagtgaataataaaatgtgtgtttgtcc

SEQ ID NO: 71 - DNA, 529 bp, Human cytomegalovirus
atgagtcccaaaaacctgacgccgttcttgacggcgttgtggctgctattgggtcacagccgcgtgccgcgggtacgcgcagaagaatgttgcgaattcataaacgtcaaccacccgccggaacgctgttacgatttcaaaatgtgcaatcgcttcaccgtcgcgctgcggtgtccggacggcgaagtctgctacagtcccgagaaaacggctgagattcgcgggatcgtcaccaccatgacccattcattgacacgccaggtcgtacacaacaaactgacgagctgcaactacaatccgttatacctcgaagctgacgggcgaatacgctgcggcaaagtgaacgacaaggcgcagtacctgctgggcgccgctggcagcgttccctatcgatggatcaacctggaatacgacaagataacccggatcgtgggcctggatcagtacctggagagcgttaagaaacacaaacggctggatgtgtgccgcgctaaaatgggctatatgctgcagtgaataataaaatgtg

SEQ ID NO: 72 - DNA, 515 bp, Human cytomegalovirus; identical to SEQ ID NO: 64

SEQ ID NO: 73 - protein, 171 aa, Human cytomegalovirus; identical to SEQ ID NO: 65

SEQ ID NO: 74 - DNA, 1977 bp, Human cytomegalovirus; identical to SEQ ID NO: 33

SEQ ID NO: 75 - DNA, 1620 bp, Human cytomegalovirus
atgcggctgtgtcgggtgtggctgtctgtttgtctgtgcgccgtggtgctgggtcagtgccagcgggagaccgcagaaaaaaaaacgattattaccgagtaccgcattactgggacgcgtgctctcgcgcgctgcctgaccaaacccgttacaagtatgtggaacagctcgtggacctcacgttgaactaccactacgatgcgagccacggcttggacaactttgacgtgctcaagagaatcaacgtgaccgaggtgtcgttgctcatcagcgactttagacgtcagaaccgtcgcggcggcaccaacaaaaggaccacgttcaacgccgccggttcgctggcgcctcacgcccggagcctcgagttcagcgtgcggctctttgccaactagcctgcgtcacgggaaataatatgctacggcttctgcttcgtcaccactttcactgcctgcttctgtgcgcggtttgggcaacgccctgtctggcgtctccgtggttcacgctaacggcgaaccagaatccgtccccgccatggtctaaactgacgtatcccaaaccgcatgacgcggcgacgttttactgtccttttctctatccctcgcccccacggtccccctcgcaattcccggggttccagcgggtattaacgggtcccgagtgtcgcaacgagaccctgtatctgctgtacaaccgggaaggccagaccttggtggagagaagctccacctgggtgaaaaaggtgatctggcatctgagcggtcgcaatcagaccatcctccaacggatgccccgaacggcttcgaaaccgagcgacggaaacgtgcagatcagcgtggaagacgccaagatttttggagcgcacatggtgcccaagcagaccaagctgctacgtttcgtcgccaacgatggcacacgttattagatgtgtgtgatgaaactggagagctgggcccacgtcttccgggactacagcgtgtcttttcaggtgcgattgacgttcaccgaggccaataaccagacttacaccttctgcacccatcccaatctcatcgtttgagcccgtcgcgcgcgcagggaattttgaaaaccgcgcgtcatgagtcccaaaaacctgacgccgttcttgacggcgttgtggctgctattgggtcacagccgcgtgccgcgggtacgcgcagaagaatgttgcgaattcataaacgtcaaccacccgccggaacgctgttacgatttcaaaatgtgcaatcgcttcaccgtcgcactgcggtgtccggacggcgaagtctgctacagtcccgagaaaacggctgagattcgcgggatcgtcaccaccatgacccattcattgacacgccaggtcgtacacaacaaactgacgagctgcaactacaatctgttatacctcgaagctgacgggcgaatacgctgcggcaaagtgaacgacaaggcgcagtacctgctgggcgccgctggcagcgttccctatcgatggatcaacctggaatacgacaagataacccggatcgtgggcctggatcagtacctggagagcgttaagaaatacaaacggctggatgtgtgccgcgctaaaatgggctatatgctgcagtgaataataaa

SEQ ID NO: 76 - DNA, 645 bp, Human cytomegalovirus
atgctacggcttctgcttcgtcaccactttcactgcctgcttctgtgcgcggtttgggcaacgccctgtctggcgtctccgtggttcacgctaacggcgaaccagaatccgtccccgccatggtctaaactgacgtatcccaaaccgcatgacgcggcgacgttttactgtccttttctctatccctcgcccccacggtccccctcgcaattcccggggttccagcgggtatcaacgggtcccgagtgtcgcaacgagaccctgtatctgctgtacaaccgggaaggccagaccttggtggagagaagctccacctgggtgaaaaaggtgatctggtatctgagcggtcgcaatcagaccatcctccaacggatgccccgaacggcttcgaaaccgagcgacggaaacgtgcagatcagcgtggaagacgccaagatttttggagcgcacatggtgcccaagcagaccaagctgctacgtttcgtcgtcaacgatggcacacgttatcagatgtgtgtgatgaaactggagagctgggcccacgtcttccgggactacagcgtgtcttttcaggtgcgattgacgttcaccgaggccaataaccagacttacaccttctgcacccatcccaatctcatcgtttga

SEQ ID NO: 77 - protein, 214 aa, Human cytomegalovirus
MLRLLLRHHFHCLLLCAVWATPCLASPWFTLTANQNPSPPWSKLTYPKPHDAATFYCPFLYPSPPRSPSQFPGFQRVSTGPECRNETLYLLYNREGQTLVERSSTWVKKVIWYLSGRNQTILQRMPRTASKPSDGNVQISVEDAKIFGAHMVPKQTKLLRFVVNDGTRYQMCVMKLESWAHVFRDYSVSFQVRLTFTEANNQTYTFCTHPNLIV

SEQ ID NO: 78 - protein, 214 aa, Human cytomegalovirus; identical to SEQ ID NO: 77

SEQ ID NO: 79 - protein, 171 aa, Human cytomegalovirus
MSPKNLTPFLTALWLLLGHSRVPRVRAEECCEFINVNHPPERCYDFKMCNRFTVALRCPDGEVCYSPEKTAEIRGIVTTMTHSLTRQVVHNKLTSCNYNPLYLEADGRIRCGKVNDKAQYLLGAAGSVPYRWINLEYDKITRIVGLDQYLESVKKHKRLDVCRAKMGYMLQ

(19) United States
(12) Patent Application Publication
(10) Pub. No.: US 2004/0110188 A1
(43) Pub. Date: Jun. 10, 2004

(54) Novel virus encoded chemokines determine the tissue tropism of human cytomegalovirus (HCMV)

(76) Inventor: Gabriele Hahn, Muenchen (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/619,189

(22) Filed: Jul. 15, 2003

(30) Foreign Application Priority Data
Jul. 16, 2002 (DE) 102 32 322.4

(51) Int. Cl. C12Q 1/70; C12Q 1/68; C12P 21/04

(52) U.S. Cl. 435/6; 435/5; 435/70.21

(57) ABSTRACT

The patent application PCT/EP02/01867 (WO 02/066629) “Recombinant vector containing infectious human cytomegalovirus genome with preserved wild-type characteristics of clinical isolates” describes the cloning of a leukotropic and endothelial cell tropic clinical isolate of human cytomegalovirus (HCMV) as a bacterial artificial chromosome (BAC) in E. coli. The cloned wild-type genome of HCMV was designated FIX-BAC (Fusion-Inducing-Factor-X)-BAC (Hahn, Khan et al., 2002). The patent application PCT/EP02/01867 (WO 02/066629) also describes the construction of virus mutants using FIX-BAC technology and subsequent phenotypical testing of virus mutants for loss of wild-type features such as leukocyte and endothelial cell tropism. The genetic determinants of endothelial cell and leukocyte tropism were assigned to the UL132-UL128 genetic locus of HCMV. Moreover, the patent application PCT/EP02/01867 (WO 02/066629) describes novel transcripts within the UL131-UL128 genetic locus which are differentially spliced. The current patent application describes in more detail the UL131-128 transcripts of clinical isolates of HCMV. Translation of the newly identified transcripts showed novel open reading frames (orfs) coding for novel putative C×C and CC chemokines which are of crucial importance for HCMV pathogenesis and tissue tropism.

### DESCRIPTION

The previous patent application PCT/EP02/01867 (WO 02/066629) describes the cloning of FIX-BAC and assigned the genetic determinants of leukocyte and endothelial cell tropism of HCMV to the UL132-UL128 genetic locus of HCMV.

In the current patent application specific virus mutants targeting individual genes within the UL131-128 region as well as at the borders of UL131-128 were constructed using a sited-directed PCR-based approach in E. coli. Recombination functions of bacteriophage λ (α/β/γ) were transiently provided on a plasmid in E. coli DH10B containing at least one copy of the FIX-BAC genome. The recombination fragment was generated by PCR over a kanamycin resistance marker (pACYC177, NEB Biolabs).

The following primers were used:

| | | | |
|---|---|---|---|
| RVFIXΔUL130-132 | (P-132-for: | 5′-AAA CCA CGT CCT CGT CAC ACG TCG TTC GCG GAC ATA GCA AGA; | [SEQ. ID NO: 1] |
| | | AAT CCA CGT CGC CAC ATC TCG AGA CGA TTT ATT CAA CAA AGC CAC | |
| | | G-3′ | |
| | P-130-rev: | 5′-AAC GGC GTC AGG TCT TTG GGA CTC ATG ACG CGC GGT TTT CAA; | [SEQ. ID NO: 2] |
| | | AAT TCC CTG CGC GCG CGA CGG GCG CCA GTG TTA CAA CCA ATT AAC | |
| | | C-3′) | |
| RVFIXΔUL130 | (P-130-for: | 5′-GCG CCA CAC GCC CGG AGC CTC GAG TTC AGC GTG CGG CTC TTT; | [SEQ. ID NO: 3] |
| | | GCC AAC TAG CCT GCG TCA CGG CGA TTT ATT CAA CAA AGC-3′ | |
| | P-130-rev: | 5′-AAC GGC GTC AGG TCT TTG GGA CTC ATG ACG CGC GGT TTT CAA; | [SEQ. ID NO: 4) |
| | | AAT TCC CTG CGC GCG CGA CGG GCG CCA GTG TTA CAA CCA ATT AAC | |
| | | C-3′ | |
| RVFIXΔ128K | (P-128-for-kons: | 5′-TGC GTT CTG TGG TGC GTC TGG ATC TGT CTC TCG ACG TTT CTG, | [SEQ. ID NO: 5] |
| | | ATA GCC ATG TTC CAT CGA CGA TTT ATT CAA CAA AGC CAC G-3′ | |
| | P-128-kons2: | 5′-CGG CAC ACA TCC AGC CGT TTG TGT TTC TTA ACG CTC TCC AGG | [SEQ. ID NO: 6]) |
| | | TAC TGA TCC AGG CCC ACG GCC AGT GTT ACA ACC AAT TAA-3′ | |
| RVFIXΔUL127 | (P-127-for: | 5′-TTG AGA TTT CTG TCG CCG ACT AAA TTC ATG TCG CGC GAT AGT, | [SEQ. ID NO: 7] |
| | | GGT GTT TAT CGC CGA TAG CGA TTT ATT CAA CAA AGC CAC-G3′ | |
| | P-127-rev: | 5′-AAT ATT GAT TTA CGC TAT ATA ACC AAT GAC TAA TAT GGC TAA; | [SEQ. ID NO: 8] |
| | | TGG CCA ATA TTG ATG CAA GCC AGT GTT ACA ACC AAT TAA-3′) | |
| RVFIXΔUL148 | (P-148-for: | 5′-GAC TAT GTG CAT GTT CGG CTA CTG AGC TAC CGA GGC GAC CCC, | [SEQ. ID NO: 9] |
| | | CTG GTC TTC AAG CAC ACT CGA TTT ATT CAA CAA AGC CAC-3′ | |
| | P-148-rev: | 5′-CAC CAG GTA GGT TAT CAA AAC GCG AGC CCA TAT CGC CGC CAT; | [SEQ. ID NO: 10) |
| | | CAT TGT AAT CAG CAA TGT GCC AGT GTT ACA ACC AAT TAA-3′ | |
| RVFIXΔUL132K | (P-132-forK: | 5′-ACG TCC TCG TCA CAC GTC GTT CGC GGA CAT AGC AAG AAA TTC, | [SEQ. ID NO: 11] |
| | | ACG TCG CCA CGT CTC GAG ACG ATT TAT TCA ACA AAG CCA-3′ | |
| | P-132-revk: | 5′-AAG GTT CTT CCA TTT CCG AGG CGG TCA GTT CAT CGT ACA CCG; | [SEQ. ID NO: 12]) |
| | | AGA CGT AGT ACC TGA TGG GGC CAG TGT TAC AAC CAA TTA ACC-3′ | |
| RVFIXΔUL132-128 | (P-131-for: | 5′-TGT CTT TCG GTT CCA ACT CTT TCC CCG CCC CAT CAC CTC GCC; | [SEQ. ID NO: 13] |
| | | TGT ACT ATG TGT CGA TTT ATT CAA CAA AGC CAC G-3′ | |
| | P-128-rev: | 5′-TCG CGC GAC ATG AAT TTA GTC GGC GAC AGA AAT CTC GAA ACG; | [SEQ. ID NO: 14]) |
| | | CGT ATT TCG GAC AAA CAC ACA TGC CAG TGT TAC AAC CAA TTA | |
| | | ACC-3′ | |
| RVFIXΔUL133-148 | (P-133-for: | 5′-CGC TGT AGG GAT AAA TAG TGC GAT GGC GTT TGT GGG AGA ACG, | [SEQ. ID NO: 15] |
| | | CAG TAG CGA TGG GTT GCG ACG TGC ACC GAT TTA TTC AAC AAA GCC | |
| | | ACG-3′ | |
| | P-148-rev: | 5′-CAC CAG GTA GGT TAT CAA AAC GCG AGC CCA TAT CGC CGC CAT; | [SEQ. ID NO: 16] |
| | | CAT TGT AAT CAG CAA TGT GCC AGT GTT ACA ACC AAT TAA-3′) | |
| RVFIXΔUL131K | (P-131-fork: | 5′-CAG TCT GCA ACA TGC GGC TGT GCT GGG TGT GGC TGT CTG TTT, | [SEQ. ID NO: 17] |
| | | GTC TGT GCG CCG TGG TGC CGA TTT ATT CAA CAA AGC CAC-3′ | |
| | P-131-rev: | 5′-GCT AGT TGG CAA AGA GCC GCA CGC TGA ACT CGA GGC TCC GGG; | [SEQ. ID NO: 18] |
| | | CGT GTG GCG GCC AGT GTT ACA ACC AAT TAA CC-3′) | |
| RVFIXΔ146-147 | (P-UL146-for: | 5′-GAT TTT CCG GGA ATA CCG GAT ATT ACG AAT TAC TGG TAG TGA, | [SEQ. ID NO: 19] |
| | | CGT AGA TAA TAA AAT TAT ACG ATT TAT TCA ACA AAG CCA CG-3′ | |
| | P-UL147-rev: | 5′-CAC CAA AGC CGT TAG CGT GCC CAG AGC TAC CGC ACG GTA AAA. | [SEQ. ID NO: 20] |
| | | TAG GGA CAT GAG CCA GTG TTA CAA CCA ATT AAC C-3′) | |

The Southern Blot was probed with the kanamycin specific probe pAcyc. The generation and testing of the mutants is described in the text. M: molecualr weight marker 1 kb ladder.

Tabel 1 summarizes the phenotypical testing of RVFIX virus mutants.

TABLE 1
Leukocyte, Monocyte and HUVEC tropism

| | PMNL-Tropism | MO-Tropism | HUVEC-Tropism |
|---|---|---|---|
| RVFIX WT | positive | positive | HUVEC+ |
| RVFIX _{Δ}UL130 | negative | negative | HUVEC− |
| RVFIX _{Δ}UL131 | negative | negative | HUVEC− |
| RVFIX _{Δ}UL132 | positive | positive | HUVEC+ |
| RVFIX _{Δ}UL128 | negative | negative | HUVEC− |
| RVFIX_{Δ}UL131-128 | negative | negative | HUVEC− |
| RVFIX_{Δ}UL127 | positive | positive | HUVEC+ |
| RVFIX_{Δ}UL148 | positive | positive | HUVEC+ |
| RVFIX_{Δ}UL133-148 | weak pos. | positive | HUVEC+ |
| RVFIX_{Δ}UL146-147 | weak pos. | positive | HUVEC+ |

PMNL: Peripheral blood mononuclear leukocytes
MO: Monocytes
HUVEC: Human umbilical vein endothelial cells

Only mutants with a deletion of either individual genes UL131, UL130, UL128 or the entire UL131-128 locus lost tropism for both endothelial cells and leukocytes. When genes next to UL131-128 were deleted (UL132, UL127 or UL148) the virus mutants were perfectly capable of infecting endothelial cells and leukocytes confirming the UL131-128 locus as the genetic determinant of endothelial cell and leukocyte tropism. When a deletion between UL133-148 was introduced into the RVFIX genome the resulting virus RVFIXΔUL133-148 retained the capability to infect endothelial cells and leukocytes, however a predominant monocyte tropism was observed. Since it is known from literature (Penfold, Dairaghi et al., 1999; Mocarski, Jr., 2002) that UL146 is a functional C×C chemokine, the predominant monocyte tropism of the virus mutant RVFIXΔUL133-148 was attributed to the loss of UL146. In order to confirm this notion, a virus mutant was constructed which UL146-UL147 was deleted. The virus mutant RVFIXΔ146-147 confirmed the RVFIXΔUL133-148 phenotype.

The role of leukocyte attraction in cytomegalovirus infection was recently underscored by the finding that, in MCMV, two differentially spliced CC chemokine homologs (Fleming, Davis-Poynter et al., 1999; MacDonald, Li et al., 1997; MacDonald, Burney et al., 1999) encoded by m131-129 (MCK-1 and MCK-2) are important determinants of virus dissemination (MacDonald, Li, & Virgin, 1997; MacDonald, Burney, Resnick, & Virgin, IV, 1999; Saederup, Lin et al., 1999; Saederup, Aguirre et al., 2001), acting as proinflammatory signals that recruit leukocytes to the site of infection to increase virus spreading. The relevance of chemoattraction is highlighted in this study by the phenotype of mutants RVΔUL146-147 and RVΔUL133-148, both bearing a functional UL131-128 locus while lacking the viral C×C chemokine genes UL146-147. UL146 product is a potent attractor and activator of human PMNL in vitro (Penfold, Dairaghi, Duke, Saederup, Mocarski, Kemble, & Schall, 1999). The mentioned virus mutants RVΔUL146-147 and RVΔUL133-148 share a full endothelial cell and monocyte tropism, but are only inefficiently transmitted to PMNL (polymorphonuclear leukocytes). These results suggest that genes within UL131-128 are sufficient both for local endothelial spread and for attraction of (and virus passage to) monocytes, while the additional chemotactic factors encoded by UL146-147 enhance virus dissemination by attracting PMNL and favouring their infection. The chemotactic activity of each individual gene product of the UL131-128 locus, as well as the cooperation with other viral or cellular gene signalling molecules, remain to be further elucidated.

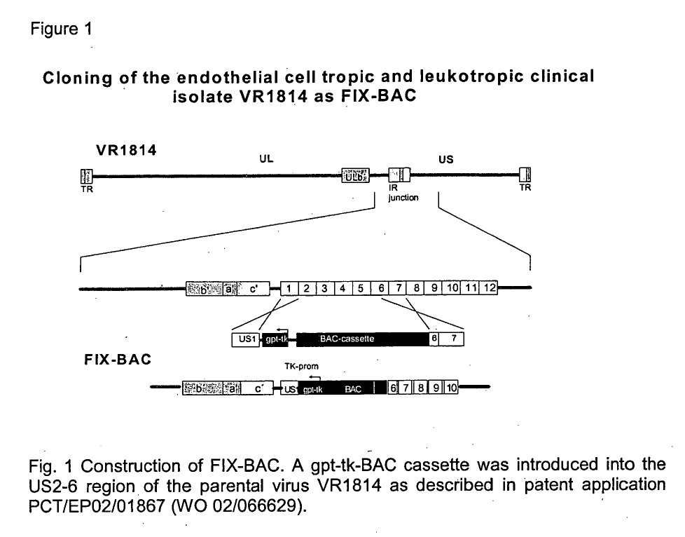

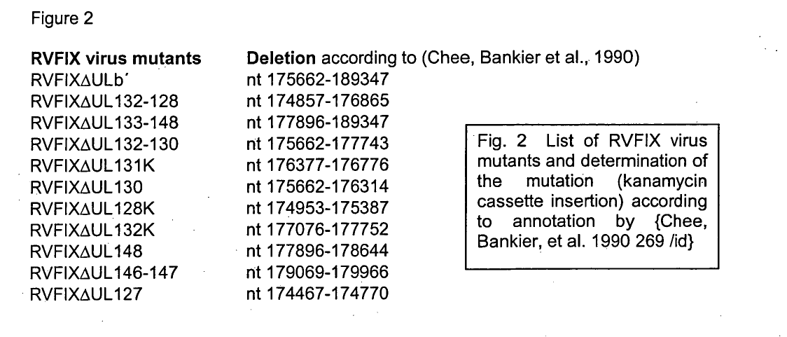

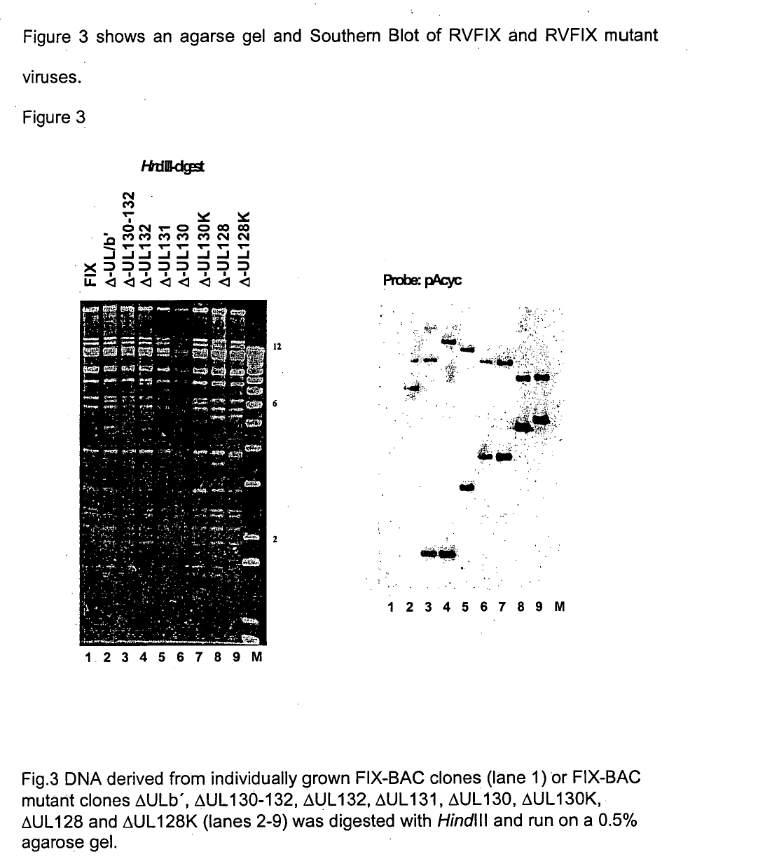

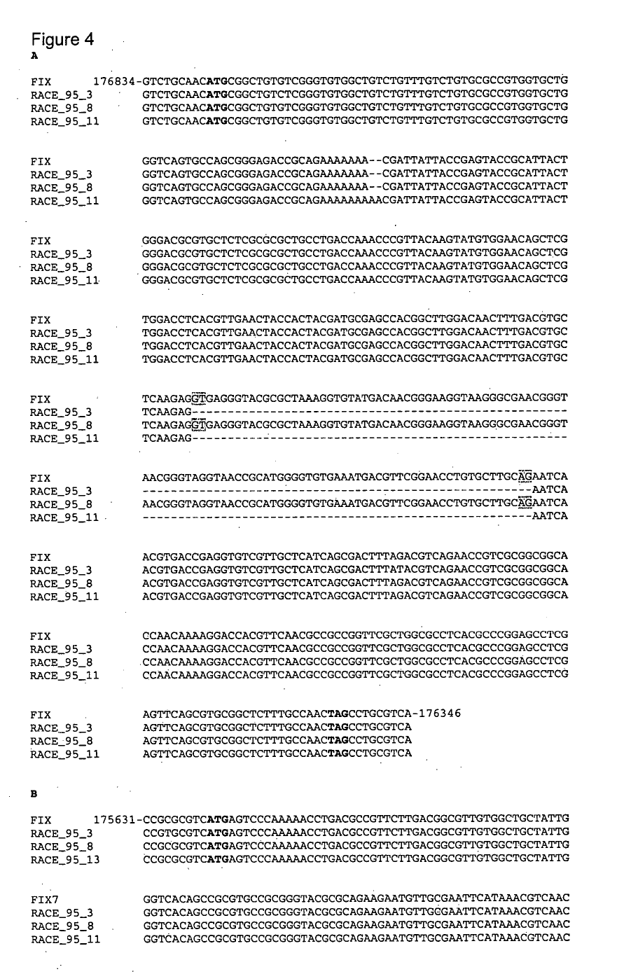

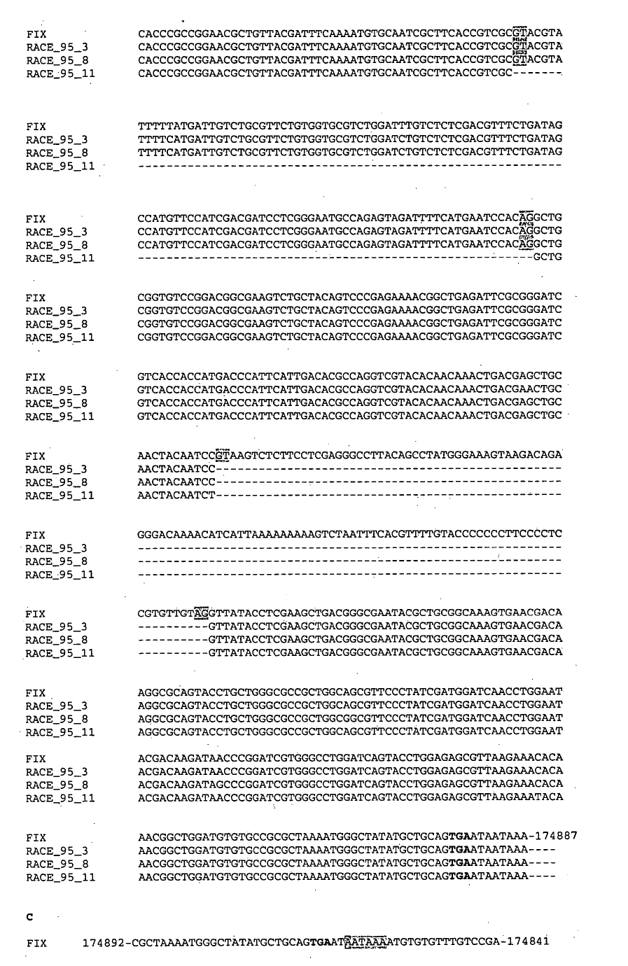

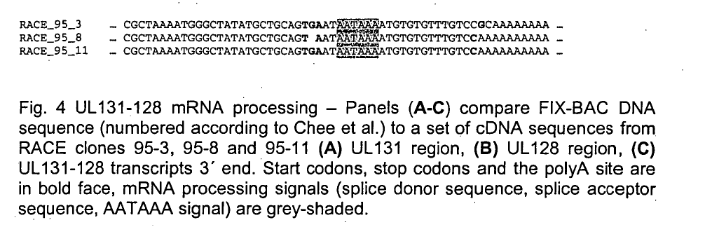

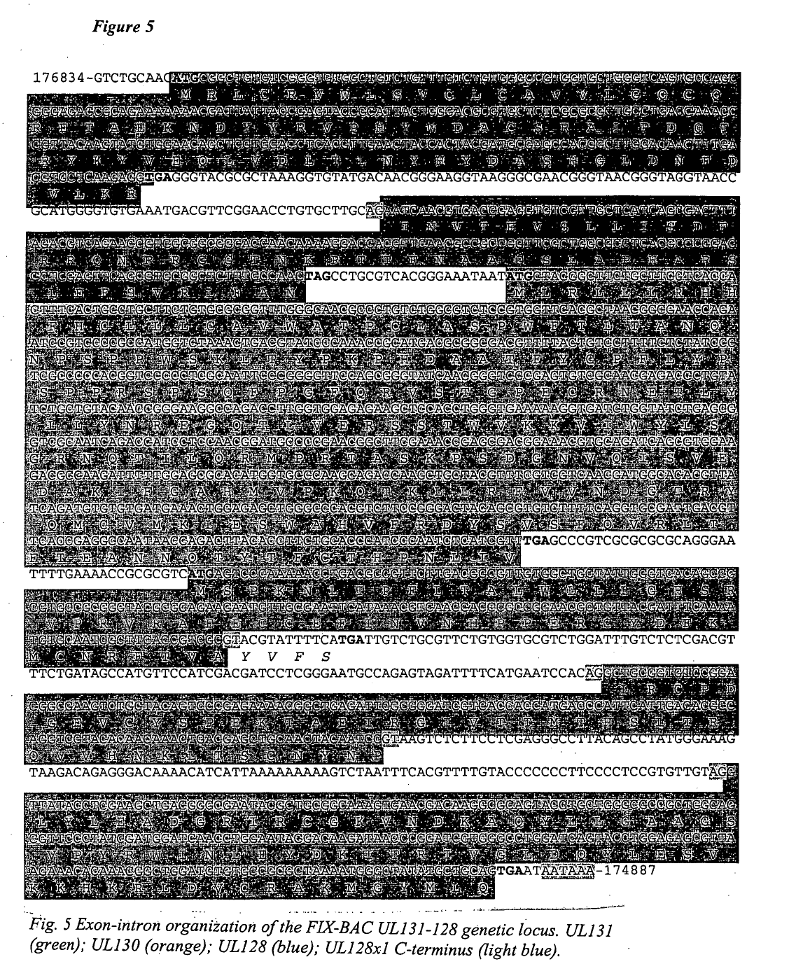

As described in patent application PCT/EP02/01867 (WO 02/066629) 5′ and 3′ RACE analyses had led to the identification of novel viral transcripts running through the genetic locus UL131-UL128 with a start codon at the beginning of UL131 and a stop codon and poly A signal at the end of UL128. The current patent application provides a more in depth analysis of the newly identified transcripts on the transcriptional and translational level. FIG. 4A-C showes a sequence comparison of the newly identified RACE clones 95-3, 95-8 and 95-11 to FIX-BAC genomic sequence. FIG. 5 shows the entire UL131-128 genetic locus and predicted individual genes.

Transcript analysis was performed by rapid amplification of cDNA ends (RACE). First strand cDNA synthesis and rapid amplification of cDNA ends (RACE) was performed using the SMART™ RACE cDNA amplification kit (Clontech) according to the manufacturer's instructions. RACE products were cloned into the pT-Adv vector (Clontech) using the AdvanTAge™ PCR cloning kit for analysis and sequencing. For rapid ampification of cDNA ends (RACE) from the 5′ RACE cDNA sample the following primers were used: primer 57-GSP1: 5′-CGG CAC ACA TCC AGC CGT TTG TGT TTC TTA 3′ [SEQ. ID NO: 35]; primer 72-GSP1: 5′-TAA CGC TCT CCA GGT ACT GAT CCA GGC CCA-3′ [SEQ. ID NO: 36]; primer 73-GSP1: 5′-TCG TCA GTT TGT TGT GTA CGA CCT GGC GTG-3′ [SEQ. ID NO: 37]; primer 74-GSP1: 5′-TAT TGG CCT CGG TGA ACG TCA ATC GCA CCT -3′ [SEQ. ID NO: 38]. For rapid ampification of cDNA ends from the 3′ RACE cDNA sample the following primers were used: primer 56-GSP2: 5′-TGT GTC GGG TGT GGC TGT CTG TTT GTC TGT-3′ [SEQ. ID NO: 39]; primer 75-GSP2: 5′-TCT GCT TCG TCA CCA CTT TCA CTG CCT GCT-3′ [SEQ. ID NO: 40]; primer 76-GSP2: 5′-CGC AGA AGA ATG TTG CGA ATT CAT AAA CGT-3′ [SEQ. ID NO: 41]; primer 77-GSP2: 5′-GCT GCG GTG TCC GGA CGG CGA AGT CTG CTA-3′ [SEQ. ID NO: 42]; primer 78-GSP2: 5′-CCA GCT GGC AGA TTC CCA AAC TAA TGA AAG-3′ [SEQ. ID NO: 43]; primer 93-GSP2: 5′-CTT TCG GTT CCA ACT CTT TCC CCG CCC CAT-3′ [SEQ. ID NO: 44]; primer 94-GSP2: 5° CAC CTC GCC TAT ACT ATG TGT ATG ATG TCT-3′ [SEQ. ID NO: 45]; primer 95-GSP2: 5′-CTC TCT TTC TCA GTC TGC AAC ATG CGG CTG-3′ [SEQ. ID NO: 46]; primer 96-GSP2: 5′-GTT GTC CAA GCC GTC GCT CGC ATC GTA GTG-3′ [SEQ. ID NO: 47]; primer 97-GSP2: 5′-CAT AAT AAA GCT CTC TTT CTC AGT CTG CAA-3′ [SEQ. ID NO: 48]; primer 98-GSP2: 5′-TAT GAT GTC TCA TAA TAA AGC TCT CTT TCT-3′ [SEQ. ID NO: 49].

Identification of Novel Tropism Determining Transcripts

The novel and previously unrecognized transcripts running through the entire UL131-128 region with a predicted UL131 start codon (nt 176825-176823 according to (Chee, Bankier, Beck, Bohni, Brown, Cerny, Horsnell, Hutchison, 111, Kouzarides, Martignetti, & ., 1990), and a UL128 stop codon (nt 174865-174863) were identified. The newly identified transcripts show a splicing event between UL128×2 and UL128×3 (nt 175201-175081), either exclusively or in conjunction with splicing between UL131×1 and UL131×2 (nt 176589-176480). An additional splicing event between UL128×1 and UL128×2 (nt 175459-175335) was observed in several clones.

3′ RACE analysis consistently identified a single polyA stretch 14±1 nucleotides 3′ to the canonical AATAAA polyA signal immediately downstream of the UL128 stop codon.

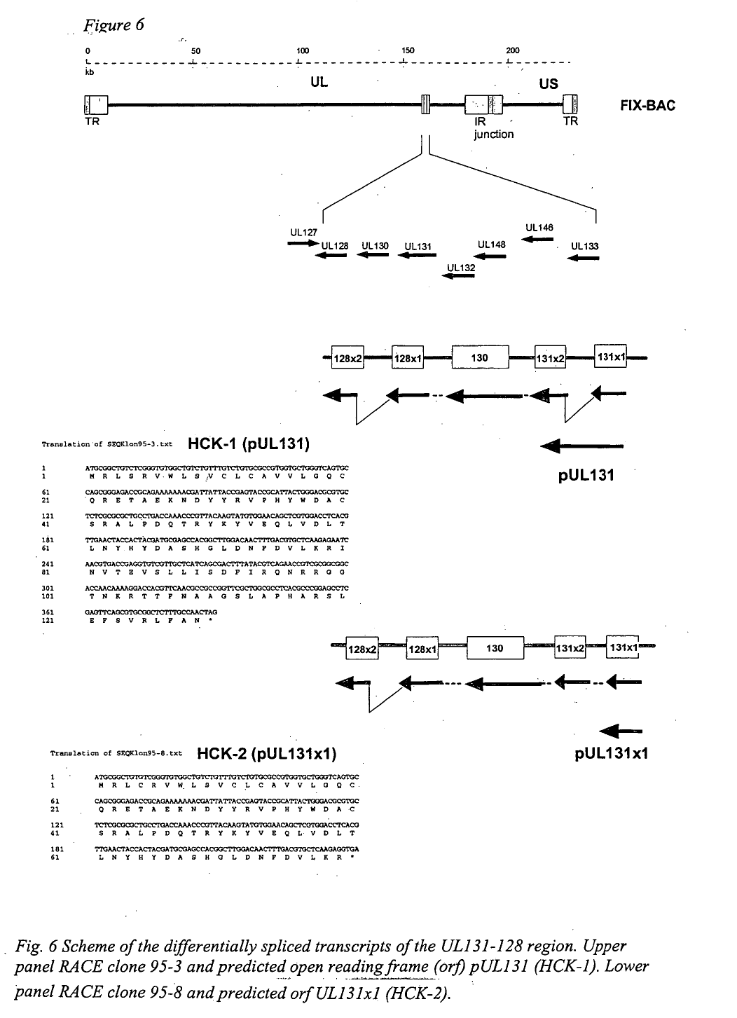

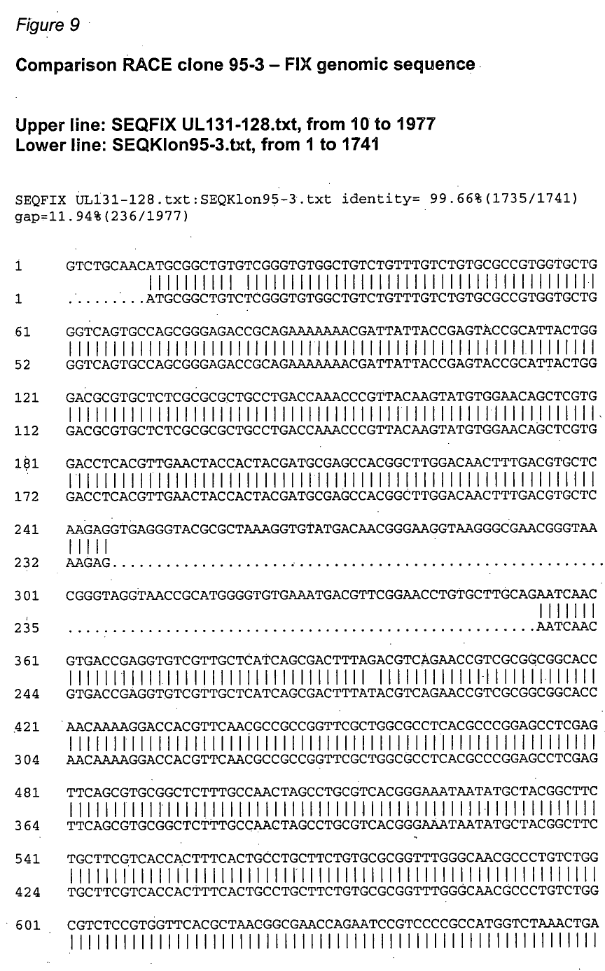

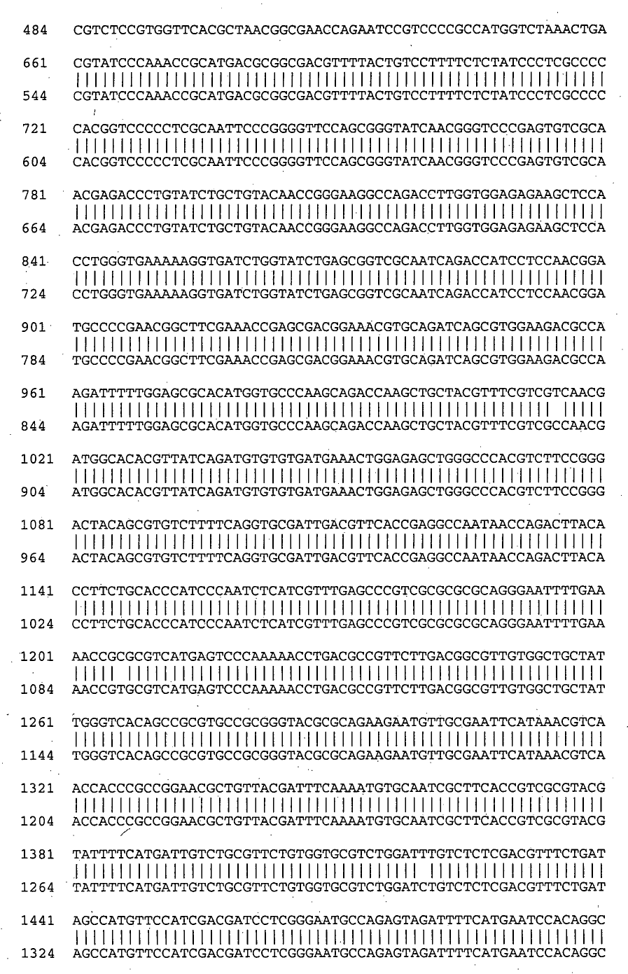

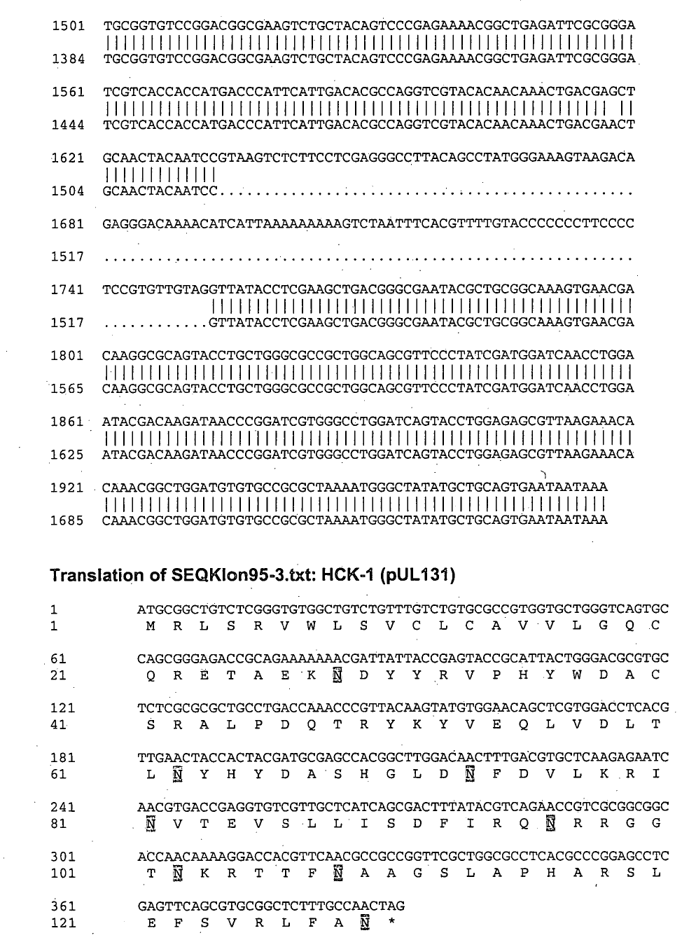

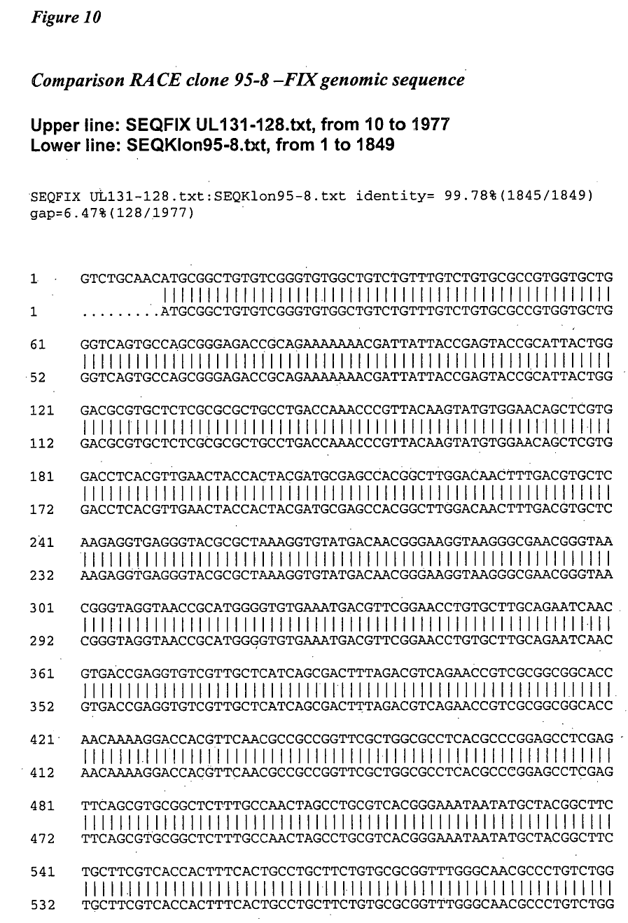

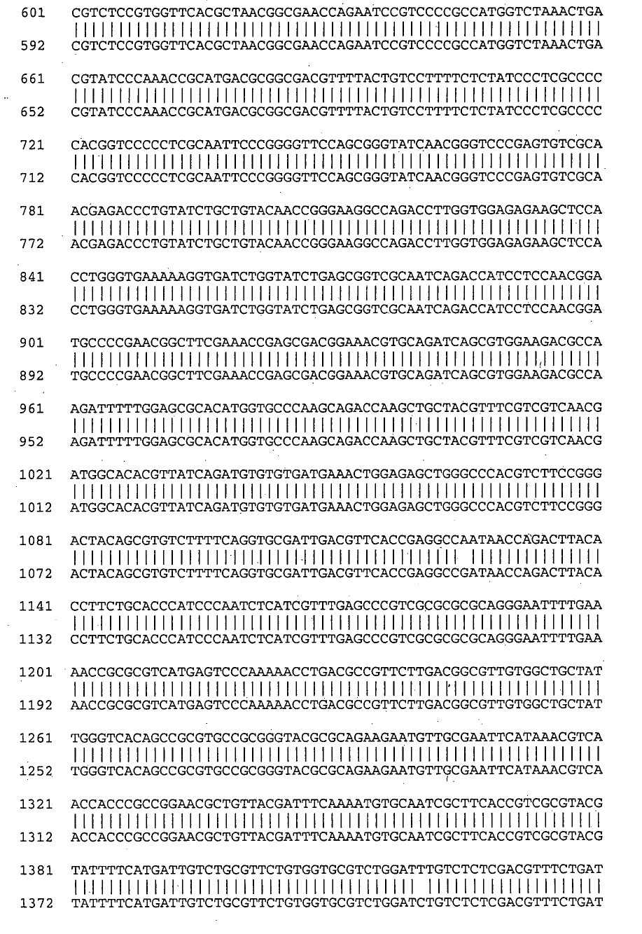

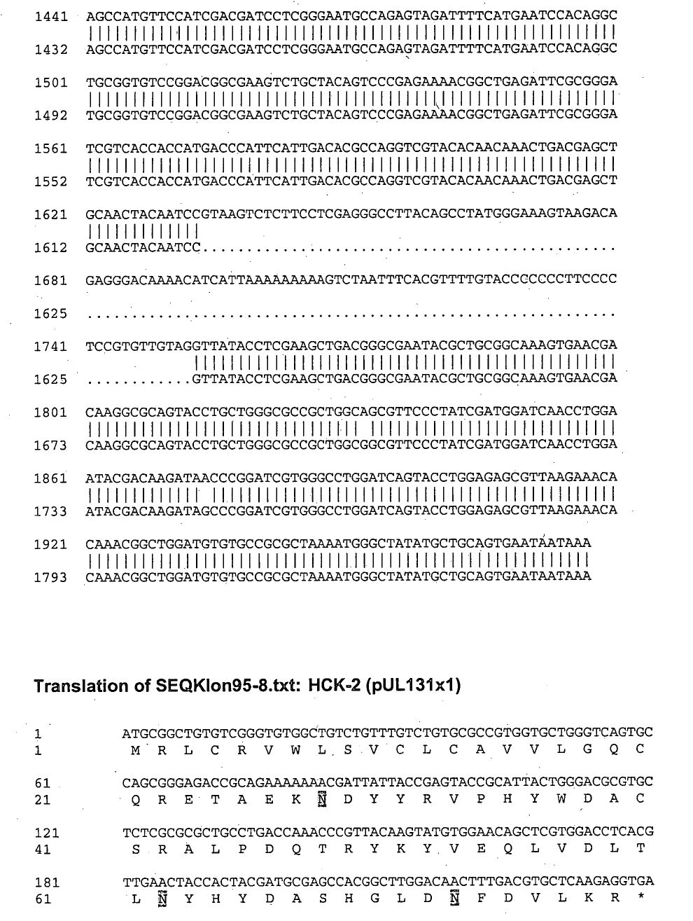

As depicted in FIG. 6 [HCK-1:nucleic acid:SEQ. ID NO: 50; amino acid: SEQ. ID NO. 51, HCK-2:nucleic acid: SEQ. ID NO: 52; amino acid: SEQ. ID NO: 53] and translation of RACE clone 95-3 (FIG. 9) [FIX:nucleic acid:SEQ. ID NO: 54; 95-3:nucleic acid:SEQ. ID NO: 55; HCK-1 nucleic acid:SEQ. ID NO: 56;amino acid:SEQ. ID NO: 57] and RACE clone 95-8 (FIG. 10) [FIX: nucleic acid:SEQ. ID NO: 58; 95-8:nucleic acid:SEQ. ID NO: 58; HCK-2:nucleic acid:SEQ. ID NO: 60; amino acid:SEQ. ID NO: 61] shows predicted proteins with a C×C motif (red) which is characteristic for C×C chemokines. RACE clone 95-3 encodes a 129 aa protein designated HCK-1 (pUL131; about 15 kDA) and RACE clone 95-8 encodes a 79 aa protein designated HCK-2 (pUL131×1; about 9 kDA). Both proteins show a number of N-linked glycosilation sides.

Both newly indentified transcripts (RACE clone 95-3 and RACE clone 95-8) have a splice between UL128×1 and UL128×2. RACE clone 95-3 has an additional splice between UL131×1 and UL131×2 which is absent in RACE clone 95-8. Sequence comparsion between the RACE clones, FIX-BAC and AD169 genomic sequences revealed that a stretch of 7×nt A (blue) in UL131 of FIX-BAC and the RACE clones 95-3 and 95-8 is extended to a stretch of 8×nt A (blue) in AD169 genomic sequence. Translation of the RACE clones 95-3 (FIG. 9) and 95-8 (FIG. 10) showed an open reading frame (ORF) with a conserved C×C C C chemokine motif (red) in FIX-BAC which is destroyed by the extension of 7×nt A to 8×nt A in the fibroblast adapted laboratory strain AD169. In RACE clone 95-3 the splice between UL131×1 and UL131×2 removes the stop codon at the end of UL131×1 and codes for a putative 129 aa C×C chemokine like protein (designated HCK-1; Human cytomegalovirus chemokine like protein), whereas in RACE clone 95-8 the stop codon at the end of UL131×1 is used to truncate the C×C chemokine like protein HCK-2 to 79 aa.

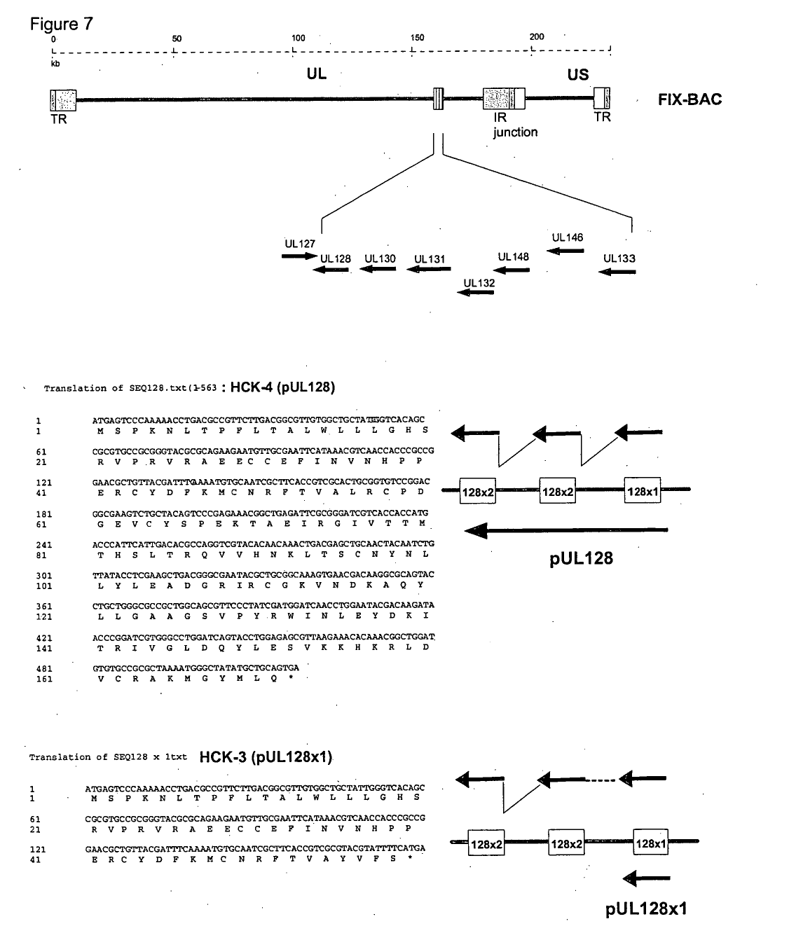

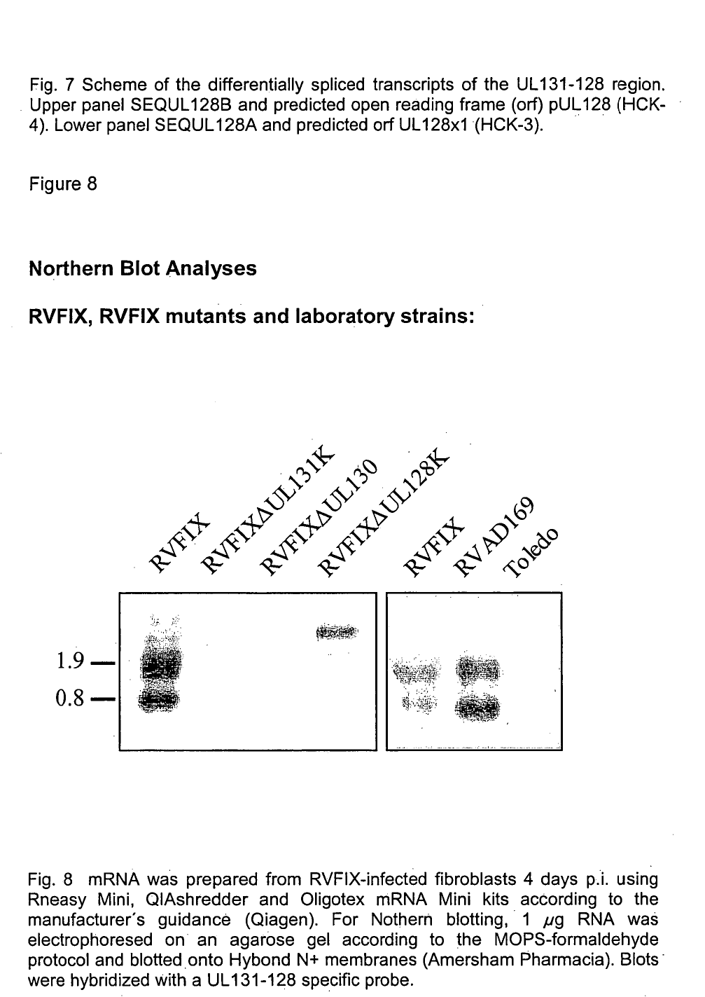

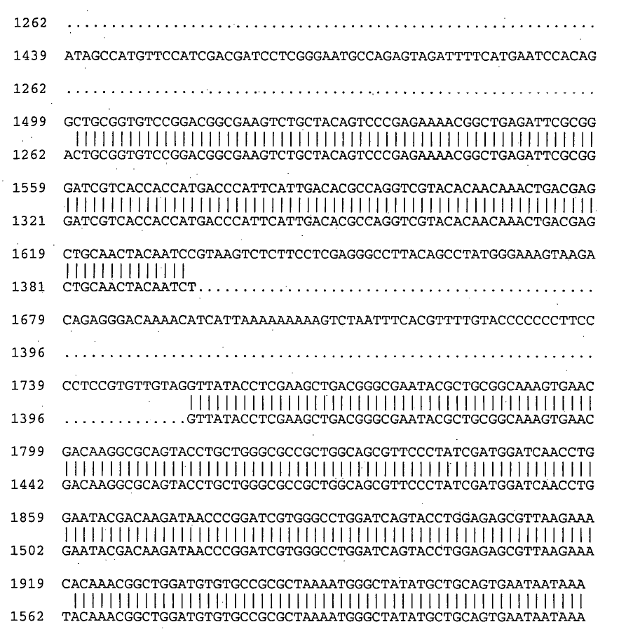

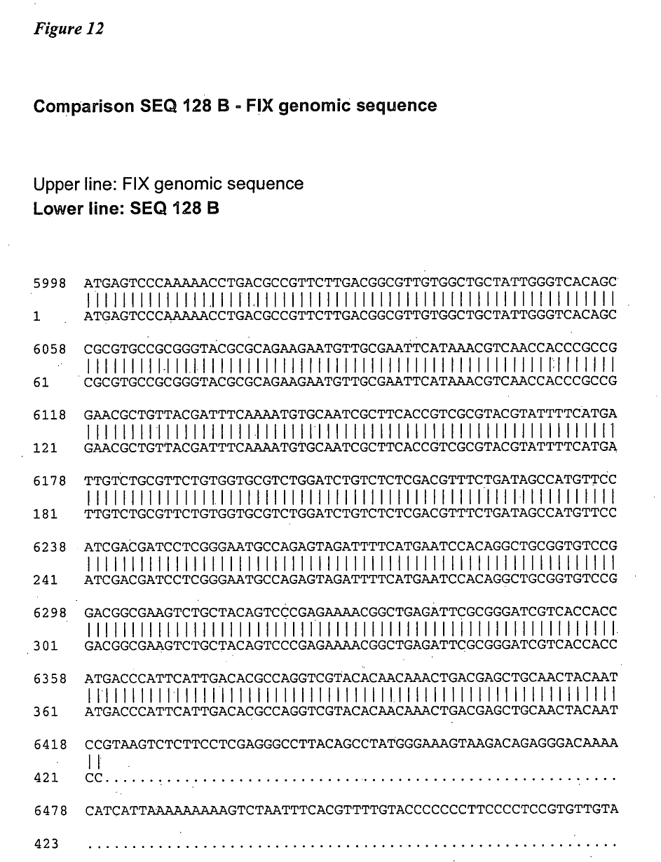

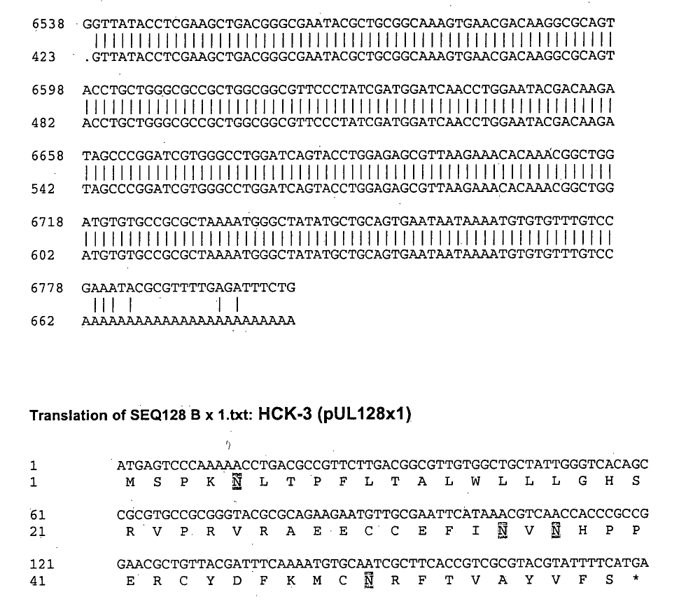

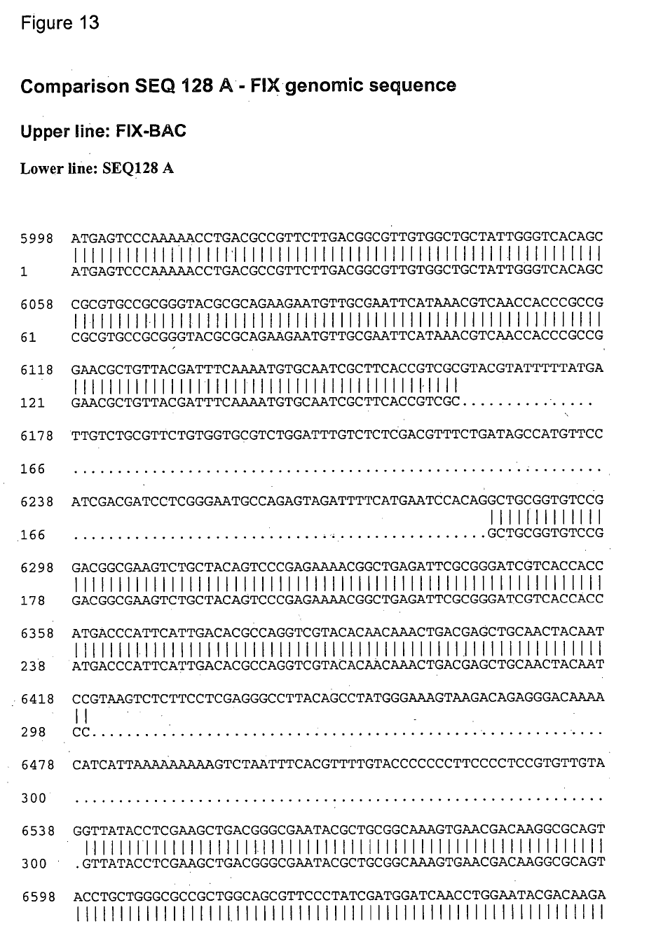

Putative CC chemokine like proteins designated HCK-3 and HCK-4 are encoded by the predicted UL128 ORF (FIG. 7) [HCK-3:nucleic acid:SEQ. ID NO: 62; amino acid:SEQ. ID NO: 63; HCK-4:nucleic acid:SEQ. ID NO: 64; amino acid:SEQ. ID NO: 65]. However, RACE analyses have not yet fully confirmed the 5′ start of these transcripts. HCK-3 (pUL128×1) (FIG. 12) [FIX:nucleic acid:SEQ. ID NO: 66; 128B:nucleic acid:SEQ. ID NO: 67; HCK-3:nucleic acid:SEQ. ID NO: 68; amino acid:SEQ. ID NO: 69]is a 59 aa protein (about 7 kDA) and HCK-4 (pUL128) (FIG. 13) [FIX:nucleic acid:SEQ. ID NO: 70; 128A:nucleic acid:SEQ. ID NO: 71; HCK-4:nucleic acid:SEQ. ID NO: 72; amino acid:SEQ. ID NO: 73] is a 171 aa protein (about 20 kDA). Splicing events between UL128×1, UL128×2 and UL128×3 fuse the three UL128 exons to form the 171 aa protein HCK-4. In case of HCK-3 the splice between UL128×1 and UL128×2 is absent and thus the TGA stop codon at the end of UL128×1 truncates the protein HCK-3 to 59 aa.

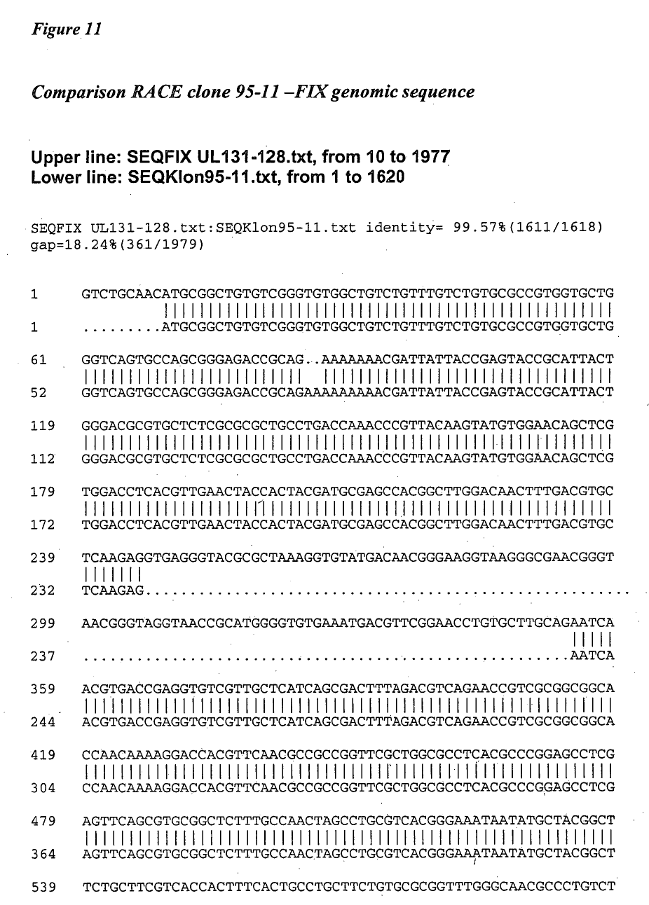

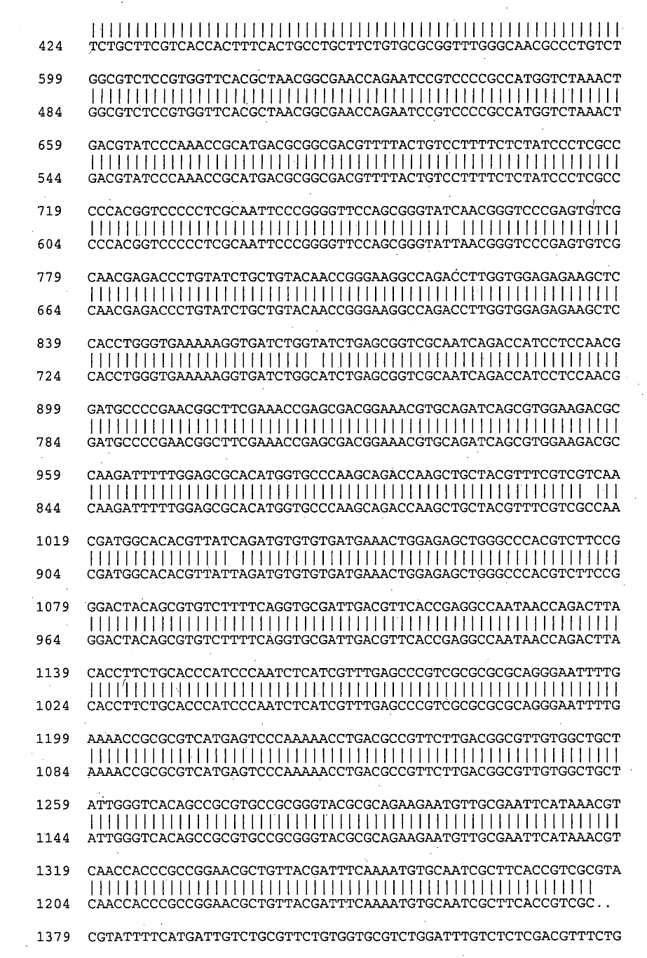

RACE clone 95-11 (FIG. 11) [FIX:SEQ. ID NO: 74; 95-11:S EQ. ID NO: 75] confirms that UL128 is composed of three exons.

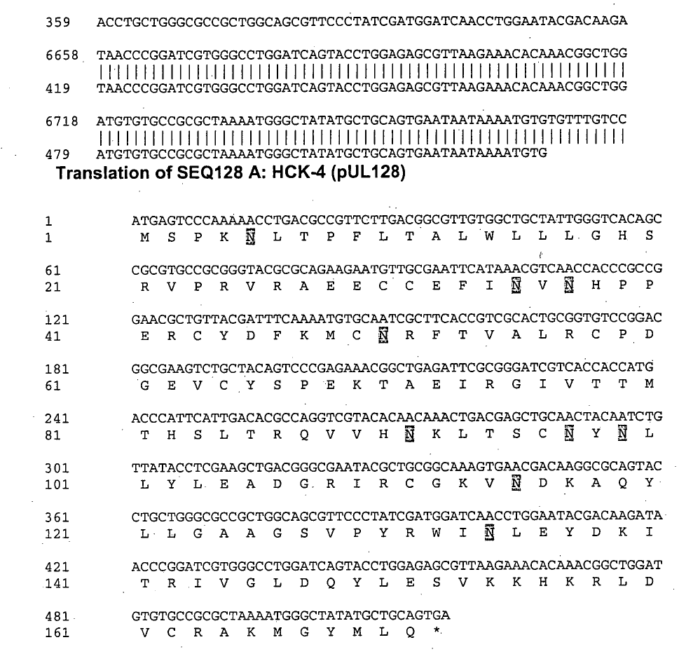

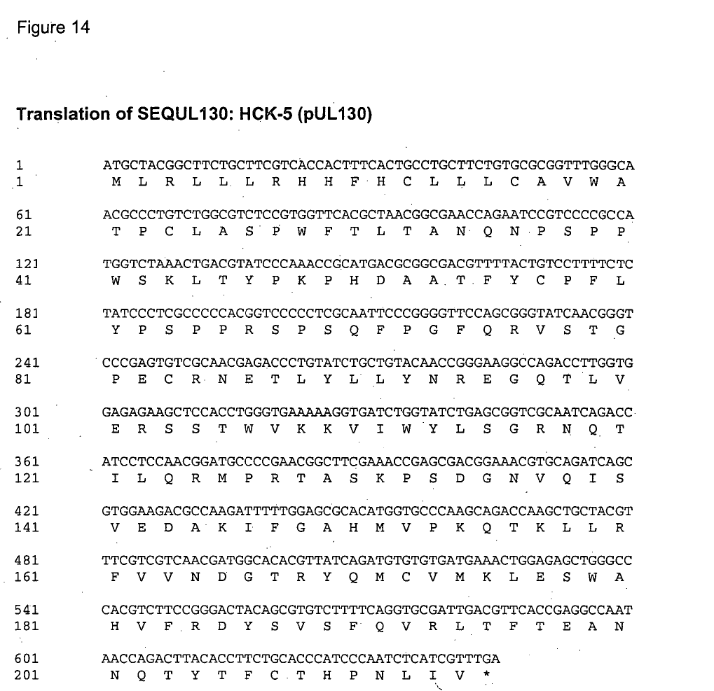

Interestingly, in RACE clone 95-11 the stretch of 7×nt A (present in RACE clones 95-3 and 95-8) is extended to a stretch of 9×nt A. This extension from 7× to 9×nt A destroys the C×C motif in RACE clone 95-11 and takes the predicted HCK-1 and HCK-2 proteins out of frame. It can be speculated that by increasing or decreasing the numbers of nt (for example nt A, nt T, nt G or nt C) in a given transcript, HCMV has found a way of transcriptional regulation of tissue tropism. It can be pictured that in a given cell type (for example fibroblasts) predominantly one transcript (for example 95-11) is synthesized over others (for example 95-8 or 95-3), thereby introducing a frame shift in an open reading frame (for example HCK-1 and HCK-2) whose product is necessary for tissue specific infection. It can also be pictured that in other cells for example endothelial cells, monocytes, leukocytes, dendritic cells or progenitor cells predominantly those transcripts are made (for example 95-3, 95-8, 128A and 128B) whose protein products (for example HCK-1, HCK-2, HCK-3 and HCK-4) encode viral chemokines and microfusion inducing factors (for example UL130, HCK-5) (FIG. 14) [nucleic acid:SEQ. ID NO: 76; amino acid:SEQ. ID NO: 77].

Closer analysis of the newly encoded proteins HCK-1 and HCK-2 shows that they have N-linked gylcosilation sites. It can be speculated that HCK-1 may be membrane bound and is trafficking through the endoplasmatic reticulum. This membrane bound HCK-1 could be of crucial importance for inducing the microfusion event (in conjunction with pUL130, HCK-5) between endothelial cells and leukocytes, monocytes, makrophages and dendritic cells or possibly other cell types. It can be assumed that HCK-2 may be a soluble chemokine which would be necessary to attract leukocytes (in conjunction with the C×C chemokines UL146 and UL147) to the site of infection. Thus HCK-1 and HCK-2 are major pathogenicity factors for virus dissemination in vivo and in vitro. It can further be assumed that the newly identified CC chemokines HCK-3 and HCK-4 may attract monocytes, macrophages, dendritic cells and possibly hematopoietic progenitor cells or stem cells to the site of infection and that concomitantly the infectious virus is spread via microfusion possibly by the use of HCK-1 and HCK-5 protein products. It can be pictured that chemokine receptors interact with the HCK-1, HCK-2, HCK-3, HCK-4 and HCK-5 protein and that the microfusion event occurs via receptor internalisation. Adhesion molecules which are upregulated by HCMV infection may provide assistance in the attachment, recruitment and microfusion process.

Transcript Analyses by Northern Blot

Northern blots from fibroblasts infected with RVFIX (FIG. 8), when hybridized with a UL131-128 specific probe, showed an upper (2.0-1.8 kb) band and a lower (0.8-0.7 kb) band. The 0.8-0.7 kb band can be interpreted as a UL128-specific transcript, the 2.0-1.8 kb band as the long UL131-128 encompassing transcript. Independent promoters may drive each transcript, one predicted upstream of the UL131 gene and the other within the UL131-130 region. The lack of a UL130 specific transcript suggests that the UL130 protein may be translated from the polycistronic 2.0-1.8 kb mRNAs through either translational reinitiation or an IRES-like mechanism.

Northern analyses of RVFIXΔUL130 or RVFIXΔUL131K infected fibroblasts shows that following the kan^{R} cassette insertion into either UL131 or UL130 the stability of all transcripts of the UL131-128 region was altered (2.0-1.8 kb transcript was absent and 0.8-0.7 kb transcript was strongly diminished), whereas kan^{R} insertion into RVFIXΔUL128 shifted both transcripts (FIG. 8). In Toledo infected cells both mRNA bands are missing as a consequence of truncation of UL128 and dislocation of the polyA signal (FIG. 8). Taken together, these data suggest that a targeted deletion of individual genes within UL131 to UL128 affects the stability of transcripts of the entire locus. Functionally, the lack of endothelial cell and leukocyte tropism observed in Toledo may correlate with the failure to express all UL131-128 transcripts. In contrast, point mutations in the UL131-128 region of the laboratory strain AD169 as compared to RVFIX neither affect mRNA mobility nor stability. However, in AD169 a stretch of 7×nt A in RVFIX is extended to 8×nt A in laboratory strain AD169. As a result, the ORF of pUL131 (HCK-1) and pUL131×1 (HCK-2) in AD169 is frameshifted.

By construction of individual virus mutants which delete individual genes UL131, UL130, UL128 it could be demonstrated that HCMV loses its tropism for monocytes, leukocytes and endothelial cells when the genetic region coding for the viral proteins HCK-1, HCK-2, HCK-3, HCK-4 or HCK-5 is removed from the virus genome. It is reasonable to assume that infection of cell types such as dendritic cells, macrophages, progenitor cells, B- and T-lymphocytes, hematopietic stem cells may occur in the same fashion and that the UL131-128 locus of HCMV and its encoded protein products described are indispensable for the infection process in vivo and in vitro.

Rescue of Leukotropism and Endothelial Cell Tropism by Ectopic Reinsertion of the ULI31-128 Region of FIX-BAC into the Laboratory Strain AD169.

In order to show that the UL131-128 region of FIX-BAC is sufficient to rescue tissue tropism of a an endothelial cell tropism and leukotropism incompetent strain, the UL131-128 genetic locus of FIX-BAC was cloned into the vector pOR16K-zeo next to an FRT site. In AD169-BAC the UL40 region was deleted and replaced with a kanamycin cassette flanked by FRT sites (from plasmid pcp015). Subsequently the kanamycin resistance marker was removed by flip recombinase (provided by plasmid pcp20) in E. coli. Ectopic insertion of the UL131-128 region from FIX-BAC (cloned into pORI6K-zeo) into AD169ΔUL40FRT-BAC was achieved by flip recombinase in E. coli and selection for kan^{R} and zeo^{R} resistant clones. The reconstituted virus RVAD169ΔUL40+UL131-128 could infect endothelial cells and leukocytes. However, the leukocyte tropism was predominantly restricted to monocytes, as expected in a virus which lacks the C×C chemokine coding genes UL146 and UL147.

The genes UL131, UL130, UL128 and the protein products with novel structure encoded (namely HCK-1, HCK-2, HCK-3, HCK-4 and HCK-5) are of fundamental importance for infection, dissemination and spread of HCMV in the human body. It can be pictured that these genes and proteins are key players in disease conditions such as vascular disease and atherosclerosis development. They are targets for drug design (small molecules, anti-sense RNA, siRNAs), anti-viral chemotherapy, vaccine development and gene therapy against HCMV and other virus induced diseases (for example HIV), as well as diseases such as cancer, autoimmune disorders and atherosclerosis. Since viruses like HCMV co-evolved with the host during human evolution it, is reasonable to assume that the newly described C×C and CC chemokines of novel structure may have as yet unidentified chemokine homologues in humans (for example secreted by human immune cells).

Reference List

Chee, M. S., Bankier, A. T., Beck, S., Bohni, R., Brown, C. M., Cerny, R., Horsnell, T., Hutchison, C. A., III, Kouzarides, T., Martignetti, J. A., and . (1990). Analysis of the protein-coding content of the sequence of human cytomegalovirus strain AD169. Curr Top Microbiol Immunol 154, 125-169.

Fleming, P., Davis-Poynter, N., Degli-Esposti, M., Densley, E., Papadimitriou, J., Shellam, G., and Farrell, H. (1999). The murine cytomegalovirus chemokine homolog, m131/129, is a determinant of viral pathogenicity. J Virol 73, 6800-6809.

Hahn, G., Khan, H., Baldanti, F., Koszinowski, U. H., Revello, M. G., and Gerna, G. (2002). The human cytomegalovirus ribonucleotide reductase homolog UL45 is dispensable for growth in endothelial cells, as determined by a BAC-cloned clinical isolate of human cytomegalovirus with preserved wild-type characteristics. J Virol 76, 9551-9555.

MacDonald, M. R., Burney, M. W., Resnick, S. B., and Virgin, H. W., IV (1999). Spliced mRNA encoding the murine cytomegalovirus chemokine homolog predicts a beta chemokine of novel structure. J Virol 73, 3682-3691.

MacDonald, M. R., Li, X. Y., and Virgin, H. W. (1997). Late expression of a beta chemokine homolog by murine cytomegalovirus. J Virol 71, 1671-1678.

Mocarski, E. S., Jr. (2002). Immunomodulation by cytomegaloviruses: manipulative strategies beyond evasion. Trends Microbiol 10, 332-339.

Penfold, M. E., Dairaghi, D. J., Duke, G. M., Saederup, N., Mocarski, E. S., Kemble, G. W., and Schall, T. J. (1999). Cytomegalovirus encodes a potent alpha chemokine. Proc Natl Acad Sci U S A 96, 9839-9844.

Saederup, N., Aguirre, S. A., Sparer, T. E., Bouley, D. M., and Mocarski, E. S. (2001). Murine cytomegalovirus CC chemokine homolog MCK-2 (m131-129) is a determinant of dissemination that increases inflammation at initial sites of infection. J Virol 75, 9966-9976.

Saederup, N., Lin, Y. C., Dairaghi, D. J., Schall, T. J., and Mocarski, E. S. (1999). Cytomegalovirus-encoded beta chemokine promotes monocyte-associated viremia in the host. Proc Natl Acad Sci U S A 96, 10881-10886.

1. Study of the genetic region UL131-128 which determines leukotropism, monocyte tropism, endothelial cell tropism in human cytomegalovirus (HCMV) in FIX-BAC and all HCMV laboratory and wild-type strains as well as BAC-cloned HCMV strains such as (TowL-BAC, HB-5-BAC, TowS-BAC, TB40E-BAC, Phoebe-BAC, Powers-BAC, AD169-BAC) and their respective reconstituted viruses.

2. Study and synthesis of the newly identified viral transcripts running through the UL131-128 genetic region which are either spliced or unspliced, sense or anti-sense and which are encoding novel C×C, CC chemokines or other attachment, fusion and cell attraction factors.

3. Study and synthesis of the newly disclosed protein products HCK-1, HCK-2, HCK-3, HCK-4 and HCK-5 as well as other potential proteins encoded by the UL132-UL128 and UL131-128 genetic region of FIX-BAC, TowL-BAC, HB-5-BAC, TowS-BAC, TB40E-BAC, Phoebe-BAC, Powers-BAC, AD169-BAC, their respective reconstituted viruses, wild-type and laboratory strains.

4. Production of monoclonal antibodies against HCK-1, HCK-2, HCK-3, HCK-4 and HCK-5, synthesis of chemotherapeutic agents interfering with HCK-1, HCK-2, HCK-3, HCK-4 and HCK-5 (for example small molecules, anti-sense RNA, siRNA).

5. Construction and study of cell lines which express or secrete HCK-1, HCK-2, HCK-3, HCK-4 and HCK-5.

6. Study of tissue tropism and pathogenesis of HCMV in vitro and in vivo by constructing virus mutants which express HCK-1, HCK-2, HCK-3, HCK-4 and HCK-5 or the newly identified transcripts (95-3, 95-8, 95-11, 128A, 128B) or other as yet unidentified transcripts of the UL132-128 or UL131-128 region.

7. Study of the transcriptional and posttranscriptional regulatory mechanisms which regulate or modify the expression of HCK-1, HCK-2, HCK-3, HCK-4 and HCK-5 or other UL132-128 or UL131-128 encoded chemokine and microfusion inducing factors regarding tissue tropism, pathogenesis of HCMV, other herpesviruses as well as DNA and RNA viruses.

8. Expression of HCK-1, HCK-2, HCK-3, HCK-4 and HCK-5 or the newly identified transcripts (95-3, 95-8, 95-11, 128A, 128B) in human or animal cells particularly immune cells in order to study or influence trafficking of such cells.

9. Use of the newly identified virus encoded chemokines HCK-1, HCK-2, HCK-3, HCK-4 and HCK-5 or the newly identified transcripts (95-3, 95-8,95-11,128A, 128B) or therapeutic agents directed against them for therapy of virus induced diseases, autoimmune disease, cancer, atherosclerosis, vasculitis, rheumatoid disease, gene therapy, vector development, vaccine development, study of trafficking and migration of leukocytes, monocytes, dendritic cells, natural killer cells, T-cells, B-cells, study of latency and reactivation of HCMV, induction or prevention of apoptosis, activation or resistance of virally infected target cells to NK cells and T cells (CTLs).

10. Study of HCK-1, HCK-2, HCK-3, HCK-4 and HCK-5 or the newly identified transcripts (95-3, 95-8, 95-11, 128A, 128B) in connection with C×C and CC chemokine receptor mediated entry of HCMV, other Herpesviruses, other DNA and RNA viruses (for example HIV) into target cells and study of cell adherence mechanisms.

11. Structural analyses of HCK-1, HCK-2, HCK-3, HCK-4 and HCK-5 and potentially other chemokine, cytokine adherence and microfusion factors encoded by the UL132-128 or UL131-128 genetic locus.

12. Study of co-infection or transfection of target cells (expressing HCK-1, HCK-2, HCK-3, HCK-4 and HCK-5) by HCMV and other DNA and RNA viruses, especially HIV virus.

13. Study of HCK-1, HCK-2, HCK-3, HCK-4 and HCK-5 or the newly identified transcripts (95-3, 95-8, 95-11, 128A, 128B) in vivo, in vitro and in animal models in connection with the development of vascular damage, development of arteriitis, vasculitis, arteriosclerosis and stenosis of the vessel wall and mechanisms of protection against such diseases.
